(19) United States
(12) Patent Application Publication
(10) Pub. No.: US 2025/0302846 A1
(43) Pub. Date: Oct. 2, 2025

(54) ANTIBIOTIC COMPOSITION AND METHODS OF USE THEREOF

(71) Applicant: THE REGENTS OF THE UNIVERSITY OF COLORADO A BODY CORPORATE, Denver, CO (US)

(72) Inventors: Corrella DETWEILER, Boulder, CO (US); Jamie DOMBACH, Louisville, CO (US)

(21) Appl. No.: 18/864,247

(22) PCT Filed: May 11, 2023
(86) PCT No.: PCT/US2023/021955

§ 371 Date: Nov. 8, 2024

Related U.S. Application Data
(60) Provisional application No. 63/340,529, filed on May 11, 2022.

(51) Int. Cl. A61K 31/551; A61P 31/04

(52) U.S. Cl. CPC A61K 31/551; A61P 31/04

(57) ABSTRACT

This disclosure generally relates to small molecule antibiotic and its novel therapeutic uses for treating bacterial infections, alone or in combination with a secondary antibiotic.

### CROSS-REFERENCE TO RELATED APPLICATIONS

This International PCT application claims the benefit of and priority to U.S. Provisional Application No. 63/340,529 filed May 11, 2022, the specification, claims and drawings of which are incorporated herein by reference in their entirety.

### GOVERNMENT INTEREST

This invention was made with government support under grant number AI151979 and AI121365 awarded by National Institutes of Health. The government has certain rights in the invention.

### TECHNICAL FIELD

This disclosure generally relates to a novel small molecule antibiotic effective against both Gram-negative and Gram-positive bacterial pathogens.

### BACKGROUND

As pathogenic bacteria become increasingly resistant to antibiotics, antimicrobials with mechanisms of action distinct from current clinical antibiotics are needed. During infection, innate immune defense molecules increase bacterial vulnerability to chemicals by permeabilizing the outer membrane and occupying efflux pumps. Therefore, screens for compounds that reduce bacterial colonization of mammalian cells have the potential to reveal unexplored therapeutic avenues. Here Applicant's describe a new small molecule, referred to herein as D66 that can act as a broad-spectrum antibiotic that can target both Gram-negative and Gram-positive bacteria. D66 inhibits bacterial growth under conditions wherein the bacterial outer membrane or efflux pumps are compromised. The compound disrupts voltage across the bacterial inner membrane at concentrations that do not permeabilize the inner membrane or lyse cells. D66 rapidly disrupts voltage across the bacterial cytoplasmic membrane of Gram-positive bacteria, and rapidly increases membrane fluidity but does not rupture the cell membrane nor affect reduction potential. Moreover, D66 further alters the Na+, K+ and Mg+ion gradients in Gram-positive bacterium.

Selection for bacterial clones resistant to D66 activity suggested that outer membrane integrity and efflux are the two major bacterial defense mechanisms against this compound.

Treatment of mammalian cells with D66 does not permeabilize the mammalian cell membrane but does cause stress, as revealed by hyperpolarization of mitochondrial membranes. Nevertheless, the compound is tolerated in mice and reduces bacterial tissue load. The current invention suggest that the inner membrane could be a viable target for anti-Gram-negative antimicrobials, and that disruption of bacterial membrane voltage without lysis is sufficient to enable clearance from the host. The current invention further suggests that D66 can target mechanosensory ion transporter in bacteria

### SUMMARY OF THE INVENTION

In one aspect, the invention includes a novel small molecule identified herein as D66 that causes the disruption of the inner membrane structure of Gram-negative bacteria, and further can disrupt the voltage across the bacterial cytoplasmic membrane of Gram-positive bacteria. These modes-of-action being generally referred to a membrane disruptor) As shown in FIG. 1, D66 is a hydrophobic small molecule containing two aromatic groups, a seven-membered saturated heterocyclic ring with two nitrogen atoms (a 1,4-diazepane), and a urea

In one preferred aspect, the membrane disruptor of the invention comprises a compound according to Formula (I), also referred to herein as D66:

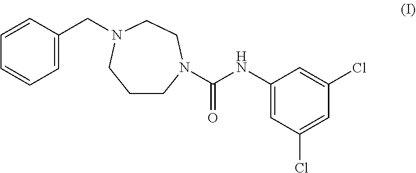 [SMILES: O=C(Nc1cc(Cl)cc(Cl)c1)N1CCCN(Cc2ccccc2)CC1]

In another preferred aspect, the compound of the invention comprises a broad-spectrum antibiotic compound capable of treating a bacterial infection caused by either a Gram-negative, or Gram-positive bacterial strain.

In another preferred aspect, the compound of the invention comprises a broad-spectrum antibiotic compounds capable of inhibiting biofilm formation caused by either a Gram-, or Gram-positive bacterial strain.

In another preferred aspect, compound of the invention can inhibit one or more mechanosensory ion transporters, which can disrupt the voltage gradient across the bacterial cytoplasmic membrane of Gram-positive bacteria. In a preferred aspect, D66 inhibition of one or more mechanosensory ion transporters can alter the Na+, K+ and Mg+ion gradients across the bacterial cytoplasmic membrane of Gram-positive bacteria.

In another preferred aspect, the compound of the invention can be co-administered with another antibiotic, which can include broad, or narrow spectrum antibiotics directed to either Gram-positive or Gram-negative pathogenic bacterial strains.

Additional aspects of the invention may become evident based on the specification and figures presented below.

### BRIEF DESCRIPTION OF THE DRAWINGS

FIG. 1A-D. D66 is a small molecule that prevents S. Typhimurium replication and/or survival in macrophages. A). Structure of D66. B) RAW264.7 macrophage-like cells were infected with S. Typhimurium harboring a chromosomal sifB: : gfp reporter, treated with DMSO or D66 at two hours after infection, and monitored for bacterial accumulation (GFP+Macrophage Area) over 16 hours using the SAFIRE (Screen for Anti-infectives using Fluorescence microscopy of Intracellular Enterobacteriaceae) assay. The IC50 value is indicated. Mean and SD of biological duplicates with technical duplicates across 9 dilutions of D66. C, D) CFU assays were performed with RAW264.7 or HeLa cells infected with S. Typhimurium SL1344 or 14028 for two hours and treated for 16 hours with DMSO or 1.5-fold dilutions of D66, prior to lysis and plating for CFU. Symbols on the Y-axes are the CFU value from DMSO-treated samples. The IC50 values are indicated. Mean and SEMs of biological triplicates with technical duplicates across 8 dilutions of D66.

FIG. 2A-E. D66 inhibits bacterial growth under conditions that damage the cell envelope and does not permeabilize the outer membrane. A, B) Dose response curves monitoring S. Typhimurium (SL1344) or E. coli (K12) growth from an OD600 of 0.01, normalized to growth in 2% DMSO under the indicated condition. Mean and SEM of at least three biological replicates performed with technical triplicates; curve fit: sigmoidal, 4P L. C-E) Checkerboard assays of S. Typhimurium SL1344 growth from an OD600 of 0.01 in LB for 18 hours with novobiocin (up to 100 μg/mL) and (C) PMB (up to 48 μM), (D) JDI (up to 150 μM), or (E) D66 (up to 150 μM). Growth was normalized to growth in 2% DMSO, with the darkest blue representing 100% growth and white representing 0%.

FIG. 3A-F. D66 rapidly perturbs the bacterial cytoplasmic membrane with minor disruption of barrier function. Mid-log phase S. Typhimurium cells grown in LB with 0.5 μg/mL PMB were used for all experiments. D66 MIC95 concentrations are provided in Table 1. A) Cell membrane potential was monitored with the fluorescent dye DiSC3 (5). Cells were treated at time 0 with DMSO, JD1 [70 μM], or D66. Data were normalized to DMSO at time 0 and corrected for the quenching effect of D66 (FIG. 6A, B) B) Cell membrane permeability was monitored by PI fluorescence. Cells were treated at time 0 with DMSO, SDS [0.005%] or D66. Samples were processed at the time points shown. A one-way ANOVA with a Dunnet's multiple comparison test, * P<0.005. C-F) Growth curves and kill-curves of cells treated at time 0 with either DMSO or D66. Culture aliquots were monitored for OD600 (C, E) or plated for enumeration of CFU (D, F). Data are presented as fold change. Mean and SEM of three biological replicates with technical triplicates are shown in all panels.

FIG. 4A-B. Analysis of resistant mutants. A-B), D66-resistant mutants selected for in an AacrAB mutant strain background. A) Diagram showing the hns mutation in six independent D66-resistant clones. B). RAW 264.7 cells were infected with S. Typhimurium SL1344, AacrAB or the six D66 resistant mutants as indicated. After 18 hours of infection, cells were lysed and plated for enumeration of CFU. Numbers above bars indicate percent of wildtype (SL1344) CFU/mL. Mean and SEM of biological triplicates with technical triplicates. A one-way ANOVA with a Dunnet's multiple comparison test: P=0.005 for AacrAB and <0.001 for all resistant mutants, compared to the wild-type strain. C) D66-resistant mutants selected for in the presence of PMB. The five independent clones identified were all resistant to PMB at concentrations 4-8X higher than the parent strain. A one-way ANOVA with a Dunnet's multiple comparison test: * P <0.05 compared to the corresponding strain without PMB.

FIG. 5A-C. D66 is well tolerated by eukaryotic cells and has antimicrobial activity in mice. A) RAW 264.7 cells were incubated with the mitochondrial membrane potential indicator TMRM, treated (red arrow) with DMSO (0.5%), CCCP, or dilutions of D66, and imaged over time. Averages and SEM of three biological replicates with technical triplicates, normalized to time 0. B) RAW 264.7 cells that were uninfected or infected with S. Typhimurium SL1344 for 2 hours were treated with DMSO or D66 and monitored for LDH release after 16 hours. Averages and SEM of three biological replicates with technical duplicates, normalized to the maximum amount of LDH release (lysed cells; % Max LDH). Symbols on the Y-axis show the percentage of LDH released by DMSO-treated cells. C) C57Bl/6 mice were intraperitoneally inoculated with S. Typhimurium. At 10 minutes and 24 hours after infection, mice were dosed with 50 mg/kg of chloramphenicol or D66 by intraperitoneal injection. Mice were euthanized 48 hours after infection. The spleen and liver were homogenized and plated for enumeration of CFU. Significance was determined by Mann-Whitney.

FIG. 6A-B. D66 quenches the fluorescent dye DiSC3 (5) in a concentration-dependent manner. A) Control wells (without bacterial cells) containing medium with 2 mM DiSC3 (5) and DMSO or compound, as indicated, added at time 0. B) Data from FIG. 3A normalized to DMSO at time 0 but without correction for the quenching effect of D66 observed in panel A. JDI was included as a control.

FIG. 7A-B. D66 pharmacokinetic parameters. Values were calculated by compartmental modeling using Phoenix WinNonlin. Data fit a two-compartment model (r=0.9976) with bolus dosing. A) Decay curve. B) Parameter values.

FIG. 8A-B. D66 inhibits the growth of Gram-positive bacteria in broth. A) Dose response curves monitoring staphylococcal and B. subtills growth in the presence of D66 from an OD600 of 0.01, normalized to growth in 2% DMSO after 18 hours. Mean and SEM of at least three biological replicates performed with technical triplicates. B) Table of D66 concentrations that inhibit growth. For comparison, S. Typhimurium is included.

FIG. 9A-B. D66 is bactericidal to mid-log phase S. aureus. A) Growth curves and B) kill-curves. Mid-log phase cultures of S. aureus FDA209 were treated at time 0 with either DMSO or the indicated concentration of D66 (1x MIC95=130 μM). Cultures were monitored for OD600 and plated for enumeration of colony forming units (CFU). Data are presented as fold change. Mean and SEM of three biological replicates performed with technical triplicates.

FIG. 10A-D. D66 rapidly disrupts voltage across the bacterial cytoplasmic membrane of Gram-positive bacteria. A) D66 quenches the fluorescent dye DiSC3 (5) in a dose-dependent manner. Data were normalized to DMSO at time 0. Relative fluorescent units (RFU). Mean and SEM of three biological replicates performed with technical triplicates. B, C) Mid-log phase S. aureus (FDA209) in LB were treated at time 0 with DMSO, gramicidin [2 μg/mL], or D66 (1x MIC95=130 μM). B) Fold change in RFU. Data are normalized to DMSO at time 0. C) Data normalized to account for the quenching effect of D66 on DiSC3 (5) observed in A. Mean and SEM of three biological replicates performed with technical triplicates. D) Bacillus subtilis expressing mNeonGreen-FtsZ at stationary phase in LB were placed on agar pads containing D66 at the indicated concentration (1x MIC95=89 μM). Confocal microscope images were taken after 15 minutes to ascertain whether D66 disrupted the septal localization of FtsZ.

FIG. 11A-E. D66 rapidly increases membrane fluidity but does not rupture the cell membrane nor affect reduction potential. Mid-log phase S. aureus FDA209 cells were used for all experiments. A) Membrane fluidity was evaluated with a Laurdan generalized polarization (GP) assay. Cells were exposed at time 0 to DMSO, benzyl alcohol (BnOH, a membrane fluidizer [50 mM]), or D66 (1x MIC95=130 μM). Mean and SEM of three biological replicates performed with technical triplicates. B) Membrane permeability was monitored by PI fluorescence. Cells were exposed at time 0 to DMSO, SDS [0.3%], or D66. Samples were processed at the timepoints shown. Mean and SEM of three biological replicates performed with technical triplicates. C) Intracellular pH was monitored with the fluorescent probe BCECF-AM. Cells were exposed at time 0 to DMSO, the protonophore CCCP [100 μM], or D66. Mean and SEM of three biological replicates performed with technical triplicates. D) Intracellular ATP levels measured using the Promega BacTiter-Glo kit after 30 minutes of treatment with DMSO, chloramphenicol (Chlor), or D66. Mean and SEM of three biological replicates performed with technical triplicates. E) Respiration rates of cells incubated with resazurin and treated at time 0 as indicated. Mean and

SEM of three biological replicates performed with technical triplicates, normalized to untreated cells at time 0.

FIG. 12A-D. Treatment with D66 did not reduce the number of viable staphylococcal persister cells in broth nor strongly decrease intracellular infection. A-D) Overnight cultures of the indicated strains were treated at time 0 with DMSO, ciprofloxacin [4 μg/mL], vancomycin [10 μg/mL], or the corresponding MIC95 concentration of D66 (1x MIC95=130 μM, 49 μg/mL). Cultures were plated for enumeration of CFU at time points indicated. Mean and SEM of three biological replicates. E) RAW 264.7 macrophage-like cells and HeLa cells were infected with S. aureus USA300, at MOIs of 0.5 and 2.5, respectively. Cells were treated two hours after infection with DMSO (gray circle on Y axis), or dilutions of D66 from 40 μM. After 8 hours of infection, cells were lysed and plated for enumeration of CFU. Mean and SEM of biological duplicates performed with technical triplicates each with six dilutions of D66. The IC50 value that was calculatable is indicated.

FIG. 13A-F. D66 treatment inhibits staphylococcal biofilm formation. Biofilm formation was monitored after 18 hours in TSB with subinhibitory concentrations of DMSO, rifampin (1x MIC95=0.05 μg/mL), vancomycin (1x MIC95=1 μg/mL), or D66 (1x MIC95=130 μM). A-D) Crystal violet staining for accumulated biofilm mass measured at A595. Mean and

SEM of three biological replicates performed with technical triplicates. E-F). Biofilms were stained with Syto 9 (live cells) and PI (damaged/dead cells) and imaged with confocal fluorescent microscopy. Volume of live, dead, and total cell volume was calculated (positive volume=positive voxel; 1 voxel=0.0367 μm3). Asterisks indicate P≤0.05 as determined by one-way ANOVA compared to DMSO. Mean and SDs derived from a minimum of four fields of view each from two biological replicates.

FIG. 14A-H. D66 treatment decreases the number of live cells in one-day-old staphylococcal biofilms. For the indicated strain, biofilms established in TSB for 24 hours were treated for 18 hours with DMSO, rifampin (1x MIC95=0.05 μg/mL), vancomycin (1x MIC95=1 μg/mL), or D66. The remaining biofilm (A-D) matrix was quantified with crystal violet or E-F) volume was quantified after Syto9 and PI staining as in FIG. 5. G, H) Syto9 and PI-stained Z-stacked images were converted to volumes. Volume data were compiled and reconstructed in Nikon Elements Advanced Research software using a color-coded volume display where blue is the bottom, lowest Z-stack and magenta is the highest point for each sample. Scale bar is in um.

Asterisks indicate P≤0.05 as determined by one-way ANOVA compared to DMSO. A-D are means and SEMs from three biological replicates performed in triplicate. E-F are means and SDs of one of two replicates derived from a minimum of four fields of view. G-H are representative images from this same replicate as in E-F.

FIG. 15A-D. D66 treatment is minimally effective at reducing 5-day-old staphylococcal biofilms. (A-D). For the indicated strain, biofilms established in TSB for 5-days were treated for 18 hours with DMSO, rifampin (1x MIC95=0.05 μg/mL), vancomycin (1x MIC95=1 μg/mL), or D66 (Table 1). The remaining biofilm extracellular matrix was stained with crystal violet and the A595 was measured. Mean and SEM of three biological replicates performed with technical triplicates are shown. Asterisks indicate P≤0.05 as determined by one-way ANOVA compared to DMSO.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

The present invention includes a novel small molecule identified herein as D66 that causes the disruption of the inner membrane structure of Gram-negative bacteria. As shown below, D66 is a hydrophobic small molecule containing two aromatic groups, a seven-membered saturated heterocyclic ring with two nitrogens (a 1,4-diazepane), and a urea.

In a preferred embodiment, the invention includes an membrane disruptor compound identified as D66 having the following structure:

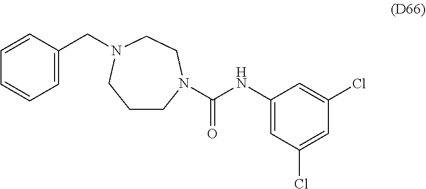 [SMILES: O=C(Nc1cc(Cl)cc(Cl)c1)N1CCCN(Cc2ccccc2)CC1]

In other preferred aspect, the compound of the invention comprises a broad-spectrum antibiotic compounds capable of treating a bacterial infection caused by either a Gram-negative, or Gram-positive bacterial strain. In other preferred aspect, the compound of the invention comprises a broad-spectrum antibiotic compounds capable of inhibiting biofilm formation caused by either a Gram-, or Gram-positive bacterial strain.

In one preferred aspect, compound of the invention can inhibit one or more mechanosensory ion transporters, which can disrupt the voltage gradient across the bacterial cytoplasmic membrane of Gram-positive bacteria. In a preferred aspect, D66 inhibition of one or more mechanosensory ion transporters can alter the Na+, K+ and Mg+ion gradients across the bacterial cytoplasmic membrane of Gram-positive bacteria.

In another preferred aspect, the compound of the invention is disclosed for treating a bacterial infection, and preferably a Gram-negative bacterial infection. In another embodiment, a method of treating a bacterial pathogen in a subject in need thereof is disclosed. The method comprises administering to the subject a therapeutically effective amount of an membrane disruptor compound, and specifically compound D66 of the invention. In a preferred embodiment, the method further comprises co-administering an antimicrobial compound to the subject in need thereof. In this embodiment, the antimicrobial compound is selected from the group consisting of: polymyxin B (PMB), and colistin, or a combination of the same. In additional embodiments, the bacterial infection comprises an infection with one or more of a Gram-negative bacteria, which may be selected from the group consisting of: Salmonella sp., K. pneumoniae, Enterobacter cloacae, Shigella sp., Neisseria sp., E. coli, or a bacterial infection caused by a Multi-Drug Resistant (MDR) Gram-negative bacteria . . .

In another embodiment, a method of treating a bacterial infection, and preferably a Gram-positive bacterial infection. . . . The method comprises contacting the cell with a therapeutically effective amount of compound D66. In another preferred embodiment, the method comprises contacting the cell with an antimicrobial compound and D66. In this embodiments, the antimicrobial compound comprises an antibiotic directed to disrupt the voltage gradient across the bacterial cytoplasmic membrane Gram-positive bacteria, which may include broad and/or narrow spectrum antibiotics that target Gram-positive bacteria. In additional embodiments, the bacterial infection comprises an infection of a cell with one or more of a Gram-positive bacteria, which may be selected from the group consisting of: staphylococcus or Bacillus.

The term “Gram-negative bacteria” as used herein refers to bacteria stained red by Gram staining and, generally, these bacteria have strong resistance to pigments and surfactants. The Gram-negative bacteria of the present invention include all types of Gram-negative bacteria containing endotoxins, and examples thereof include, but are not limited to, bacteria belonging to the genus Escherichia, the genus Pseudomonas, the genus Acinetobacter, the genus Salmonella, the genus Klebsiella, the genus Neisseria, the genus Enterobacter, the genus Shigella, the genus Moraxella, the genus Helicobacter, the genus Stenotrophomonas, the genus Bdellovibrio, and the genus Legionella. In particular, examples of these Gram-negative bacteria include, but are not limited to, Escherichia coli, Pseudomonas aeruginosa, Pseudomonas fluorescens, Pseudomonas putida, Pseudomonas chlororaphis, Pseudomonas pertucinogena, Pseudomonas stutzeri, Pseudomonas syringae, Acinetobacter baumannii, Acinetobacter lwoffii, Acinetobacter calcoaceticus, Acinetobacter haemolyticus, Salmonella enterica, Salmonella bongori, Salmonella enteritidis, Salmonella typhimurium, Salmonella gallinarum, Salmonella pullorum, Salmonella mbandaka, Salmonella choleraesuls, Salmonella thompson, Salmonella infantis, Salmonella derby, Klebsiella pneumonia, Klebsiella granulomatis, Klebsiella oxytoca, Klebsiella terrigena, Neisseria gonorrhoeae, Neisseria meningitidis, Enterobacter aerogenes, Enterobacter cloacae, Shigella boydii, Shigella dysenteriae, Shigella flexneri, Shigella sonnei, Moraxella catarrhalis, Moraxella lacunata, Moraxella bovis, Helicobacter pylori, Helicobacter heilmannii, Helicobacter felis, Helicobacter mustelae, Helicobacter fenelliae, Helicobacter rappini, Helicobacter hepaticus, Helicobacter bilis, Helicobacter pullorum, Stenotrophomonas maltophilia, Stenotrophomonas nitritireducens, Bdellovibrio bacteriovorus, Legionella pneumophila, Legionella anisa, Legionella birminghamensis, Legionella bozemanii, Legionella cincinnatiensis, Legionella dumoffii, Legionella feeleii, Legionella gormanii, Legionella hackeliae, Legionella israelensis, Legionella jordanis, Legionella lansingensis, Legionella longbeachae, Legionella maceachernii, Legionella micdadei, Legionella oakridgensis, Legionella sainthelensi, Legionella tucsonensis, and Legionella wadsworthii.

As used herein, the term “Gram positive bacteria” means bacteria that have an inner and outer membrane, and a thin peptidoglycan layer. Gram positive bacteria type, species, or genera of bacteria that, when exposed to the Gram stain, retains the dye and is, thus, stained blue-purple. Gram-positive bacteria include, but are not limited to, Bacillus, Geobacillus, Clostridium, Streptococcus, Cellulomonas, Corynebacterium, Lactobacillis, Lactococcus, Oenococcus and Eubacterium.

Other representative Gram positive bacteria include bacteria in the Enterococcus genus, including but not limited to, E. faecalis (also known as Group D Streptococcus) and E. faecium. Representative Gram positive bacteria include, but are not limited to, bacteria in the Micrococcaceae family. Gram positive bacteria in the Micrococcaceae family include, but are not limited to, bacteria in the Staphylococcus genus, including S. epidermidis,. S. aureus, S. auricularis, S. capitis, S. caprae, S. cohnii, S. epidermidis, S. felis, S. haemolyticus, S. hominis, S. intermedius, S. lugdunensis, S. pettenkoferi, S. saprophyticus, S. schleiferi, S. simulans, S. vitulus, S. warneri, and S. xylosus.

Representative acid-fast Gram positive bacteria include, but are not limited to, bacteria in the Mycobacteriaceae family. Acid-fast Gram positive bacteria in the Mycobacteriaceae family include, but are not limited to, bacteria in the Mycobacterium genus, such as M. bovis species and M. tuberculosis species. Representative members of the Mycobacterium genus include: M. abscessus, M. africanum, M. agri, M. aichiense, M. alvei, M. arupense, M. asiaticum, M. aubagnense, M. aurum, M. austroafricanum, Mycobacterium avium complex (MAC), including, M. avium, M. avium paratuberculosis, M. avium silvaticum, M. avium “hominissuis,” M. boenickei, M. bohemicum, M. bolletii, M. botniense, M. bovis, M. branderi. M. brisbanense, M. brumae, M. canariasense, M. caprae, M. celatum, M. chelonae, M. chimaera, M. chitae, M. chlorophenolicum, M. chubuense, M. colombiense, M. conceptionense, M. confluentis, M. conspicuum, M. cookii, M. cosmeticum, M. diernhoferi, M. doricum, M. duvalii, M. elephantis, M. fallax, M. farcinogenes, M. flavescens, M. florentinum, M. fluoroanthenivorans, M. fortuitum, M. fortuitum subsp. acetamidolyticum, M. frederiksbergense, M. gadium, M. gastri, M. genavense, M. gilvum, M. goodii, M. gordonae, M. haemophilus, M. hassiacum, M. heckeshornense, M. heidelbergense, M. hiberniae, M. hodleri, M. holsaticum, M. houstonense, M. immunogenum, M. interjectum, M. intermedium, M. intracellulare, M. kansasii, M. komossense, M. kubicae, M. kumamotonense, M. lacus, M. lentiflavum, M. leprae, M. lepraemurium, M. madagascariense, M. mageritense, M. malmoense, M. marinum, M. massiliense, M. microti, M. monacense, M. montefiorense, M. moriokaense, M. mucogenicum, M. muraie, M. nebraskense, M. neoaurum, M. neworleansense, M. nonchromogenicum, M. novocastrense, M. obuense, M. palustre, M. parafortuitum, M. parascrofulaceum, M. parmense, M. peregrinum, M. phlei, M. phocaicum, M. pinnipedii, M. porcinum, M. poriferae, M. pseudoshottsii, M. pulveris, M. psychrotolerans, M. pyrenivorans, M. rhodesiae, M. saskatchewanense, M. scrofulaceum, M. senegalense, M. seoulense, M. septicum, M. shimoidei, M. shottsii, M. simiae, M. smegmatis, M. sphagni, M. szulgai, M. terrae, M. thermoresistibile, M. tokaiense, M. triplex, M. triviale, Mycobacterium tuberculosis complex (MTBC), including M. tuberculosis, M. bovis, M. bovis BCG, M. africanum, M. canetti, M. caprae, M. pinnipedii′, M. tusciae, M. uicerans, M. vaccae, M. vanbaalenii, M. wolinskyi, and M. xenopi.

The compound of the invention can be administered to a patient or a subject to achieve a desired physiological effect. Generally, the subject is an animal, typically a mammal, and preferably a human. The compound can be administered in a variety of forms adapted to the chosen route of administration, i.e., orally, or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

In one embodiment, the compound D66 comprises a pharmaceutical composition. In another embodiment, the compound D66 and one or more antibiotics also comprises a pharmaceutical composition.

A “pharmaceutical composition” or “pharmaceutical composition of the invention” refers to a compound of the invention or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional antibiotic, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

The term “pharmaceutically acceptable carrier” as used herein further pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. Suitable carriers, diluents, excipients, etc. can be found in standard pharmaceutical texts. See, for example, “Handbook of Pharmaceutical Additives,” 2nd Edition (eds. M. Ash and I. Ash), 2001 (Synapse Information Resources, Inc., Endicott, N.Y., USA), “Remington's Pharmaceutical Sciences “, 20th edition, pub. Lippincott, Williams & Wilkins, 2000; and “Handbook of Pharmaceutical Excipients “, 2nd edition, 1994.

Suitable pharmaceutically acceptable carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition of the invention may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages. Exemplary parenteral administration forms include solutions or suspensions of active compounds in sterile aqueous solutions, for example, aqueous propylene glycol or dextrose solutions. Such dosage forms may be suitably buffered, if desired.

A pharmaceutical composition of the invention may be administered as single or multiple agents, for example a pharmaceutical composition of a the compound of the invention, or a pharmaceutical composition of the compound of the invention and an antibiotic compound. In some embodiments, the methods include one or more of the following effects: (1) treating a Gram-negative bacterial infection in a subject; (2) inhibiting growth of Gram-negative bacteria; (3) preventing infection of a Gram-negative bacterial infection in a subject; and (4) sensitizing or re-sensitizing a Gram-negative bacterial infection to an antibiotic. Pharmaceutical compositions suitable for the delivery of the compound of the invention as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Typical compositions or preparations according to the invention are prepared such that an oral dosage unit form contains from about 1 to about 1000 mg of active compound.

Pharmaceutical compositions for use in the methods of the present invention may be prepared by any of the methods of pharmacy, but all methods include the step of bringing the active ingredient into association with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent.

The compound of the invention can also be administered parenterally. Solutions of the active compound as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the compound of the invention in the required amount in the appropriate solvent with various other ingredients enumerated above, as required, followed by filtered sterilization. Generally, dispersions are prepared by incorporating the various sterilized ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The compound of the invention can be administered to a subject alone or in combination with pharmaceutically acceptable carriers, as noted above, the proportion of which is determined by the solubility and chemical nature of the compound, chosen route of administration and standard pharmaceutical practice.

The physician can readily determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

A “therapeutically effective amount” means the amount of a compound that, when administered to a subject for treating a bacterial infection, is sufficient to effect such treatment for the bacterial infection. The “therapeutically effective amount” will vary depending on the compound, the disease and its severity and the age, weight, etc., of the mammal to be treated. “Treating” or “treatment” of a bacterial infection includes: (1) preventing the bacterial infection, i.e., causing the clinical symptoms of a bacterial infection not to develop in a subject that may be exposed to or predisposed to the infection but does not yet experience or display symptoms of the infection; (2) inhibiting the bacterial infection, i.e., arresting or reducing the development of the infection or its clinical symptoms; or (3) relieving the bacterial infection, i.e., causing regression of the infection or its clinical symptoms.

As noted above, the compound of the invention, or pharmaceutical composition comprising the compound of the invention, may be administered to a “subject,” and preferably a human subject, by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. The subject may be a eukaryote, an animal, a vertebrate animal, a mammal, a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), an ape (e.g., gorilla, chimpanzee, orangutang, gibbon), or a human.

It will be appreciated that appropriate dosages of the active compound, and compositions comprising the active compound, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In certain embodiments, the compound disclosed herein may be administered to treat a bacterial infection to a subject in need thereof. In certain embodiments, the compound of the invention may be administered for a prophylactic treatment, or for a therapeutic treatment. In embodiments, the method of administration varies depending on the bacteria involved and the severity of the infection. Dosing regimens may vary based upon the condition being treated and the method of administration. In embodiments, the subject is given a therapeutically effective amount of the compound. An effective amount is the amount required to treat or prevent a bacterial infection as described herein. The compound of the invention may be mixed with a suitable carrier substance. In embodiments, the compound is mixed with the suitable carrier substance in an amount of 1-99% by weight of the total weight of the composition. In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

In preferred embodiments, a pharmaceutical composition of the invention comprises a therapeutically effective amount of compound D66, and may further include a therapeutically effective amount of an antibiotic that disrupts the outer membrane of a Gram-negative bacteria. In this embodiment, the antibiotic is selected from: polymyxin B (PMB), and colistin, or a combination of the same. In alternative embodiments, a pharmaceutical composition of the invention comprises a therapeutically effective amount of compound D66, and may further include a therapeutically effective amount of an antibiotic selected from: penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, o×y tetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, peril oxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, garenoxacin, ramoplanin, faropenem, polymyxin, tigecycline, AZD2563, and trimethoprim.

In alternative embodiments, a pharmaceutical composition of the invention comprises a therapeutically effective amount of compound D66, and may further include a therapeutically effective amount of an antibiotic directed to Gram-positive bacteria, selected from the class of antibiotics: Penicillins, penicillinase resistant Cephalosporins, Macrolides (Erythromycin, Clarithromycin, Azithromycin, Quinolones, Vancomycin, Sulfonamide/trimethoprim, Clindamycin, Tetracyclines, and Chloramphenicol.

Bacterial infections include any bacterial infection caused by or associated with Gram-negative bacteria, but are not limited to, bacterial pneumonia, urinary tract infections, intra-abdominal infections, skin and skin structure infections, bone and joint infections, central nervous center infections, gastro-intestinal tract infections, pelvic inflammatory diseases. Diseases associated with bacterial infections, include, but are not limited to rheumatoid arthritis, fibromyalgia, autonomic nervous dysfunction, multiple sclerosis, interstitial cystitis, multiple sclerosis, and chronic fatigue.

### Definitions

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compound are within the scope of the invention. Unless otherwise stated, all tautomeric forms of the compound of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C-or 14C-enriched carbon are within the scope of this invention. Such compounds are useful, for example, as analytical tools, as probes in biological assays, or as therapeutic agents in accordance with the present invention. The term “stereoisomer” refers to a molecule that is an enantiomer, diastereomer or geometric isomer of a molecule. Stereoisomers, unlike structural isomers, do not differ with respect to the number and types of atoms in the molecule's structure but with respect to the spatial arrangement of the molecule's atoms. Examples of stereoisomers include the (+) and (−) forms of optically active molecules.

As used herein the singular forms “a,” “an,” and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including,” as well as other related forms, such as “includes” and “included,” is not limiting.

The term “about” as used herein is a flexible word with a meaning similar to “approximately” or “nearly.” The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1% compared to the specifically recited value.

The term “compound,” or “composition,” or “compound of the invention” includes all solvates, complexes, polymorphs, radiolabeled derivatives, tautomers, stereoisomers, and optical isomers of the novel compound of the invention having a structure according to the structure of D66, and salts thereof, unless otherwise specified.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included.

“Pharmaceutical compositions” are compositions that include an amount (for example, a unit dosage) of the compound of the invention together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in

Remington's Pharmaceutical Sciences, Mack Publishing Co., Easton, Pa. (19th Edition). The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The term “pharmaceutically acceptable salt” means a salt which is acceptable for administration to a patient, such as a mammal, such as human (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compound include those wherein the compound is protonated by an inorganic or organic acid to form a cation, with the conjugate base of the inorganic or organic acid as the anionic component of the salt. For therapeutic use, salts of the compound are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned above are meant to comprise the therapeutically active non-toxic acid and base addition salt forms which the compound can form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic, and like acids. Conversely, these salt forms can be converted into the free base form by treatment with an appropriate base. The compound containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g., the lithium, sodium, potassium, magnesium, calcium salts and the like, salts with organic bases, e.g., the benzathine, N-methyl-D-glucamine, hydrabamine salts, and salts with amino acids such as, for example, arginine, lysine, and the like.

“Heterocyclyl,” or “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1, 3] dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, and 1,1 dioxo thiomorpholinyl. Unless stated otherwise specifically in the specification, a heterocyclyl group can be optionally substituted.

The term “aromatic” as applied to cyclic groups refers to ring structures which contain double bonds that are conjugated around the entire ring structure, possibly through a heteroatom such as an oxygen atom or a nitrogen atom. Aryl groups, pyridyl groups and furan groups are examples of aromatic groups. The conjugated system of an aromatic group contains a characteristic number of electrons, for example, 6 or 10 electrons that occupy the electronic orbitals making up the conjugated system, which are typically un-hybridized p-orbitals.

The term “aryl” or “aromatic moiety” as used herein refers to an aromatic ring system, which may further include one or more non-carbon atoms. These are typically 5-6 membered isolated rings, or 8-10 membered bicyclic groups, and can be substituted. Thus, contemplated aryl groups include (e.g., phenyl, naphthyl, etc.) and pyridyl. Further contemplated aryl groups may be fused (i.e., covalently bound with 2 atoms on the first aromatic ring) with one or two 5- or 6-membered aryl or heterocyclic group and are thus termed “fused aryl” or “fused aromatic “.

Aromatic groups containing one or more heteroatoms (typically N, O or S) as ring members can be referred to as heteroaryl or heteroaromatic groups. Typical heteroaromatic groups include monocyclic C5-C6 aromatic groups such as pyridyl, pyrimidyl, pyrazinyl, thienyl, furanyl, pyrrolyl, pyrazolyl, thiazolyl, oxazolyl, isothiazolyl, isoxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, pyrazolopyrimidyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. As also used herein, the terms “heterocycle,” “cycloheteroalkyl,” and “heterocyclic moieties” are used interchangeably herein and refer to any compound in which a plurality of atoms form a ring via a plurality of covalent bonds, wherein the ring includes at least one atom other than a carbon atom as a ring member. Particularly contemplated heterocyclic rings include 5- and 6-membered rings with nitrogen, sulfur, or oxygen as the non-carbon atom (e.g., imidazole, pyrrole, triazole, dihydropyrimidine, indole, pyridine, thiazole, tetrazole etc.). Typically, these rings contain 0-1 oxygen or sulfur atoms, at least one and typically 2-3 carbon atoms, and up to four nitrogen atoms as ring members. Further contemplated heterocycles may be fused (i.e., covalently bound with two atoms on the first heterocyclic ring) to one or two carbocyclic rings or heterocycles and are thus termed “fused heterocycle” or “fused heterocyclic ring” or “fused heterocyclic moieties” as used herein. Where the ring is aromatic, these can be referred to herein as ‘heteroaryl’ or heteroaromatic groups.

The term “substituted” as used herein refers to a replacement of a hydrogen atom of the unsubstituted group with a functional group, and particularly contemplated functional groups include nucleophilic groups (e.g.,—NH2,—OH,—SH,—CN, etc.), electrophilic groups (e.g., C (O) OR, C (X) OH, etc.), polar groups (e.g.,—OH), non-polar groups (e.g., heterocycle, aryl, alkyl, alkenyl, alkynyl, etc.), ionic groups (e.g.,—NH3+), and halogens (e.g., F,—Cl),

NHCOR, NHCONH2, OCH2COOH, OCH2CONH2, OCH2CONHR, NHCH2COOH, NHCH2CONH2, NHSO2R, OCH2-heterocycles, PO3H, SO3H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties. In addition to the disclosure herein, in a certain embodiment, a group that is substituted has 1, 2, 3, or 4 substituents, 1, 2, or 3 substituents, 1 or 2 substituents, or 1 substituent.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### Examples

### Example 1: Overview and summary of inventive features and therapeutic applications of D66

Here the present inventors describe a new small molecule, D66, that prevents the survival of a human Gram-negative pathogen in macrophages. D66 inhibits bacterial growth under conditions wherein the bacterial outer membrane or efflux pumps are compromised, but not in standard microbiological media. The compound disrupts voltage across the bacterial inner membrane at concentrations that do not permeabilize the inner membrane or lyse cells. Selection for bacterial clones resistant to D66 activity suggested that outer membrane integrity and efflux are the two major bacterial defense mechanisms against this compound. Treatment of mammalian cells with D66 does not permeabilize the mammalian cell membrane but does cause stress, as revealed by hyperpolarization of mitochondrial membranes. Nevertheless, the compound is tolerated in mice and reduces bacterial tissue load. These data suggest that the inner membrane could be a viable target for anti-Gram-negative antimicrobials, and that disruption of bacterial membrane voltage without lysis is sufficient to enable clearance from the host.

The effect of D66 on bacteria

Bacteria are assaulted by host soluble innate immune defenses in all body fluids, including in serum, the contents of phagolysosomes, and the cytosol. Therefore, small molecules that are unable to breach the Gram-negative cell envelope in standard microbiological media may be able to gain access to bacteria during infection. These compounds could be identified by their ability to prevent bacterial survival during infection, as within the SAFIRE assay. D66 and JD1 appear to be examples of such molecules because they enable bacterial killing in macrophages and in animals but only under broth conditions that compromise the outer membrane and/or efflux pumps. These observations further establish that Gram-negative bacteria are protected from the compounds by a combination of their outer membrane and efflux pumps. Both compounds inhibit S. Typhimurium growth in MIC assays, which utilize cells recovering from stationary phase at low cell density (OD 0.01). Both compounds also disrupt voltage across the bacterial inner membrane of mid-log phase cells (OD 0.4-0.6). However, only JD1 rapidly permeabilizes the inner membrane and kills bacteria: at 1x MIC, PI signal increases 33-fold within 30 minutes of JD1 treatment, compared to two-fold with D66. D66 therefore appears to have a more subtle effect, requiring higher concentrations and/or more time to permeabilize the inner membrane, and the bacterial cells recover over time from the damage wrought by D66. It is feasible that D66 is effectively diluted by a higher density of bacteria and/or that mid-log phase cells are more resistant to the compound than cells recovering from stationary phase. It is also possible that one or both compounds have unknown additional effects on the bacteria, the host cell, or both. Nevertheless, both D66 and JD1 appear to interact directly with bacterial cells in the context of cell envelope damage and to attack the inner membrane, suggesting that the inner membrane is vulnerable to small molecules during infection and that voltage disruption may be sufficient to augment bacterial killing by innate immune defenses and enable an intracellular pathogen to be eliminated by the host.

Potentiation of D66 by PMB likely reflects PMB permeabilization of the outer membrane

In Klebsiella pneumoniae, AcrAB-TolC contributes to resistance to PMB, indicating that PMB is an AcrAB-TolC substrate. If both D66 and PMB are exported by AcrAB-TolC, then potentiation of D66 by PMB could reflect competition for efflux. However, in E. coli and S. Typhimurium, PMB resistance is mediated primarily by LPS modifications, and likely at high PMB concentrations by the MdtEF-TolC efflux pump, which accumulates in E. coli upon PMB treatment. Therefore, the simplest explanation for the observation that PMB potentiated D66 to inhibit bacterial growth is that PMB increases outer membrane permeability and D66 access to the bacterial cell.

Bacteria appear to resist D66 based on a combination of outer membrane integrity and efflux pumps

Resistance to D66 in broth was selected for in an AacrAB mutant background and in the presence of PMB. The six independent D66-resistant clones recovered in a AacrAB background had predicted loss-of-function mutations in the gene encoding H-NS, which increases the expression of efflux pump genes, including acrEF, acrD, mdtEF, macAB, and emrKY. The five independent clones recovered from selection in the presence of PMB and D66 had increased resistance to PMB. Thus, Applicants were not successful at using a selection-for-resistance strategy to identify potential D66 target pathways. It follows that analysis of resistant mutants may not be the most efficient approach for gleaning mechanism of action for compounds that need help traversing the outer membrane and/or appear to be efflux pump exported. Nonetheless, these observations reveal that bacteria normally protect themselves from D66 based on a combination of outer membrane integrity and export of the compound through efflux pumps.

The mammalian host and D66

In cell culture infection experiments, D66 was active against S. Typhimurium in macrophages. However, for both bacterial strains, the compound had little effect on bacterial load in Hela cells for reasons that are not understood. Applicants speculate that the microenvironment of the macrophage phagosome is more effective at permeabilizing the bacterial outer membrane and/or could modify the compound to increase potency. D66 reduced viable bacteria in macrophages and in mice without obvious tolerability issues in uninfected or infected animals. However, uninfected macrophages do respond to D66 treatment, as revealed by mitochondrial hyperpolarization, an indicator of cell stress. Mitochondrial membranes use multiple, complex compensation mechanisms to increase membrane voltage over time in response to depolarization. For instance, hyperpolarization occurs upon treatment with agents that interfere with oxidative phosphorylation, ATP synthase, or proton consumption. Therefore, hyperpolarization may not be due to a primary effect of D66 on this organelle. In addition, the lack of obvious murine pathology upon D66 exposure suggests that damage caused by D66 is minimal and/or that cells recover. Overall, the modest effects noted for this compound on mammalian cells and whole animals suggests there is value in exploring the use of compounds that target bacterial inner membranes as antibacterial compounds.

### Example 2: A small molecule prevents S. Typhimurium survival in macrophages

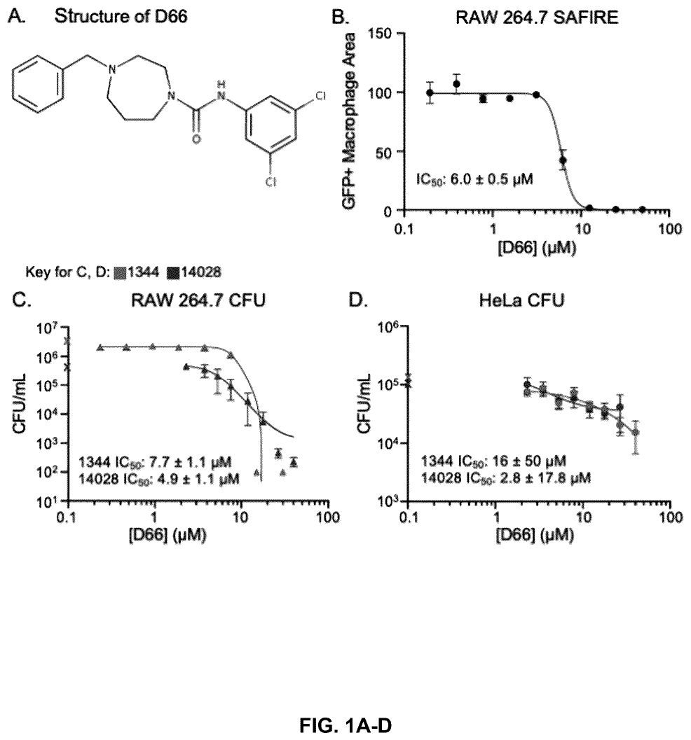

D66 is a hydrophobic small molecule (cLogP of 4.73, 378 g/mol) that contains two aromatic groups, a seven-membered saturated heterocyclic ring with two nitrogens (a 1,4-diazepane), and a urea (FIG. 1A). This compound, which has not been previously studied, was found to be highly active in a high content screening platform known as SAFIRE (Screen for Anti-infectives using Fluorescence microscopy of Intracellular Enterobacteriaceae). SAFIRE reports the accumulation of S. Typhimurium within macrophages based on sifB: : gfp expression. Here Applicants found that the half maximum inhibitory concentration (IC50) for GFP signal in macrophages was 6.0=0.5 μM (FIG. 1B). To establish whether D66 reduces bacterial load or interferes with GFP expression, Applicants lysed infected macrophages that had been treated with the compound for 16 hours and plated for bacterial colony forming units (CFU). The CFU IC50S of two virulent S. Typhimurium strains were 7.7+1.1 μM (SL1344) and 4.9+1.1 μM (14028) (FIG. 1C). Since S. Typhimurium can replicate in other cell types, Applicants also tested the ability of D66 to reduce bacterial load in Hela cells, which are derived from epithelial cells. The compound had little effect; while the calculated ICsos are low, the 5-10-fold reduction in CFU, compared to the >1000-fold CFU reduction in macrophages does not give confidence that D66 is highly active in Hela cells (FIG. 1D). These data demonstrate that D66 enables the killing of intracellular S. Typhimurium in macrophages.

### Example 3: D66 inhibits bacterial growth under conditions that compromise the cell envelope

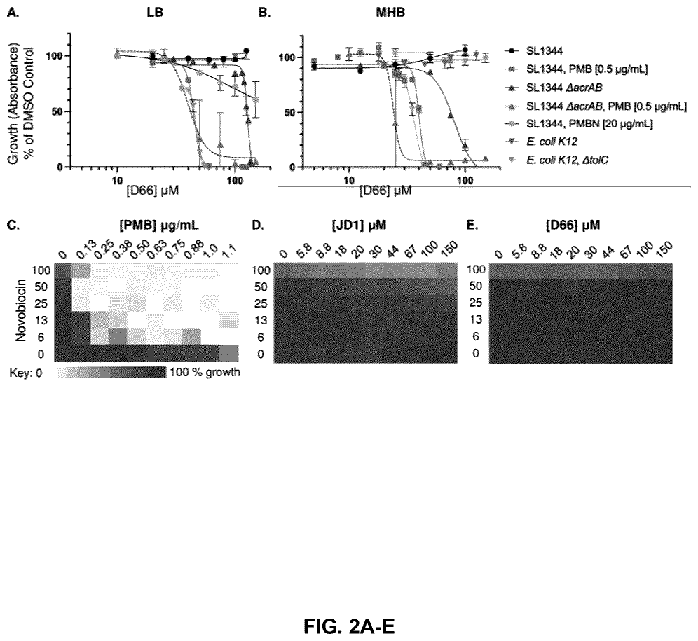

To determine whether D66 could act directly on bacteria, Applicants exposed bacteria to the compound under standard broth conditions, in lysogeny broth (LB) or cation-adjusted Mueller-Hinton Broth (MHB) (Table 1, FIG. 2A, B). No inhibition of re-growth from stationary phase was observed, consistent with previous compounds identified with the SAFIRE assay. However, under conditions that compromise the LPS layer of the outer membrane and/or efflux pumps, D66 prevented growth. Specifically, in the presence of the CAMP polymyxin B (PMB), D66 had a calculated minimum inhibitory concentration 95 (cMIC95, defined as the concentration at which 95% of growth of the corresponding strain was inhibited) of 54 μM. In these experiments, polymyxin B was at a sublethal concentration [0.5 μg/mL], which Applicants previously showed permeabilizes the S. Typhimurium outer, but not inner, membrane. In contrast to PMB, the polymyxin B nonapeptide (PMBN) did not potentiate D66: concentrations of PMBN [20 μg/mL] that in MHB enable novobiocin to reach cellular targets did not enable D66 to inhibit growth. Both PMB and PMBN bind LPS, but the latter lacks the fatty acid tail and is less disruptive to the outer membrane. Applicants also found that strains lacking genes encoding efflux pump subunits (acrAB or tolC) are sensitive to D66, compared to the parent strains. This effect that was stronger in MHB than in LB (Table 1, FIG. 2B). These data indicate that D66 slowly traverses an intact outer membrane and is captured and expelled by efflux pumps. Outer membrane permeabilization or loss of efflux pump subunits thus facilitate D66 antimicrobial activity. D66 therefore has a direct, negative effect on bacterial growth under conditions that compromise the outer membrane and/or efflux pumps.

### Example 4: The outer membrane does not appear to be permeabilized by D66

The sensitivity of the AacrAB and AtolC mutants to D66 suggests that D66 needs to cross the outer membrane to mediate its effect. However, if D66 were to damage the outer membrane and thereby facilitate its own entry and that of PMB into the cell, this could explain why the compound is potentiated by both PMB and by mutations in efflux pumps. Applicants therefore established whether treatment with D66 potentiates growth inhibition with novobiocin, an antibiotic that cannot traverse the outer membrane. Control compounds included PMB and JD1, which permeabilizes inner membranes and is not expected to potentiate novobiocin (FIG. 2 C, D). D66 did not potentiate novobiocin at concentrations up to 150 μM (FIG. 2 E). These data indicate that D66 does not appear to permeabilize the outer membrane, indicating it inhibits growth by an alternative mechanism(s).

### Example 5: D66 disrupts voltage without permeabilizing the bacterial inner membrane

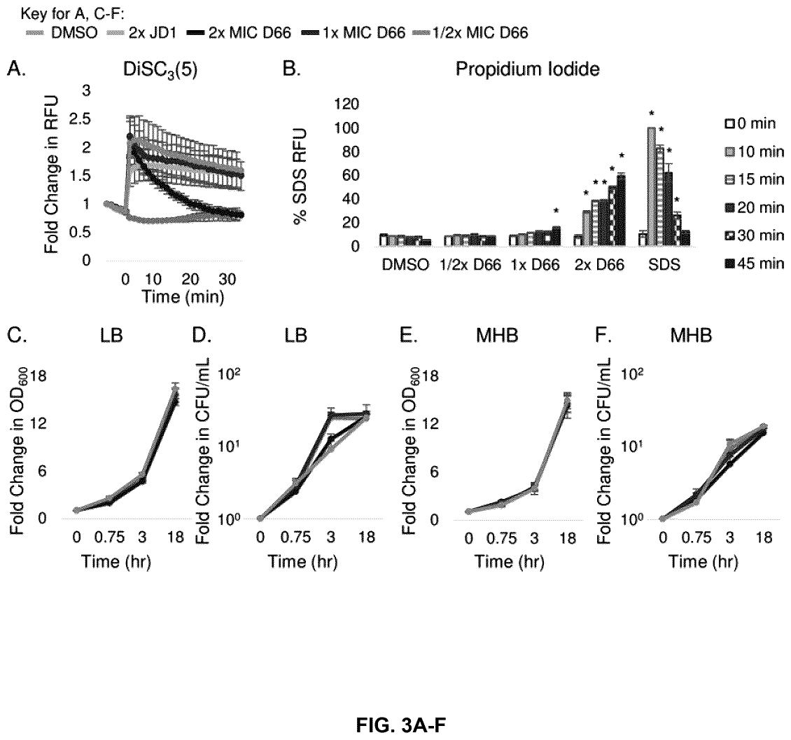

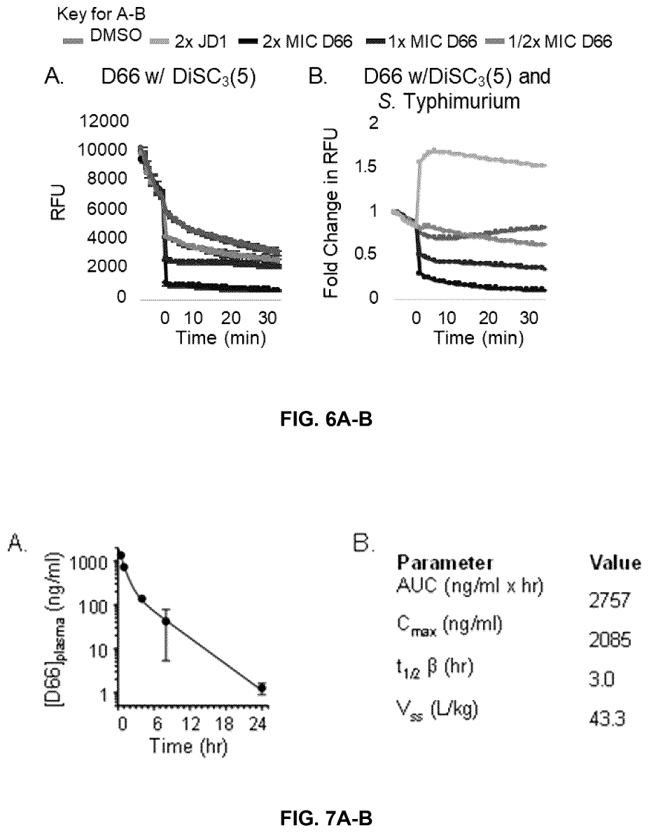

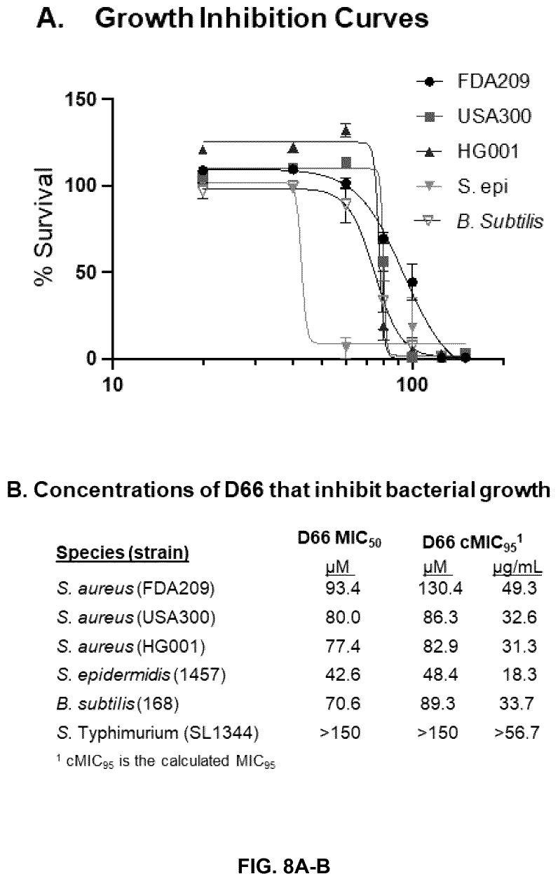

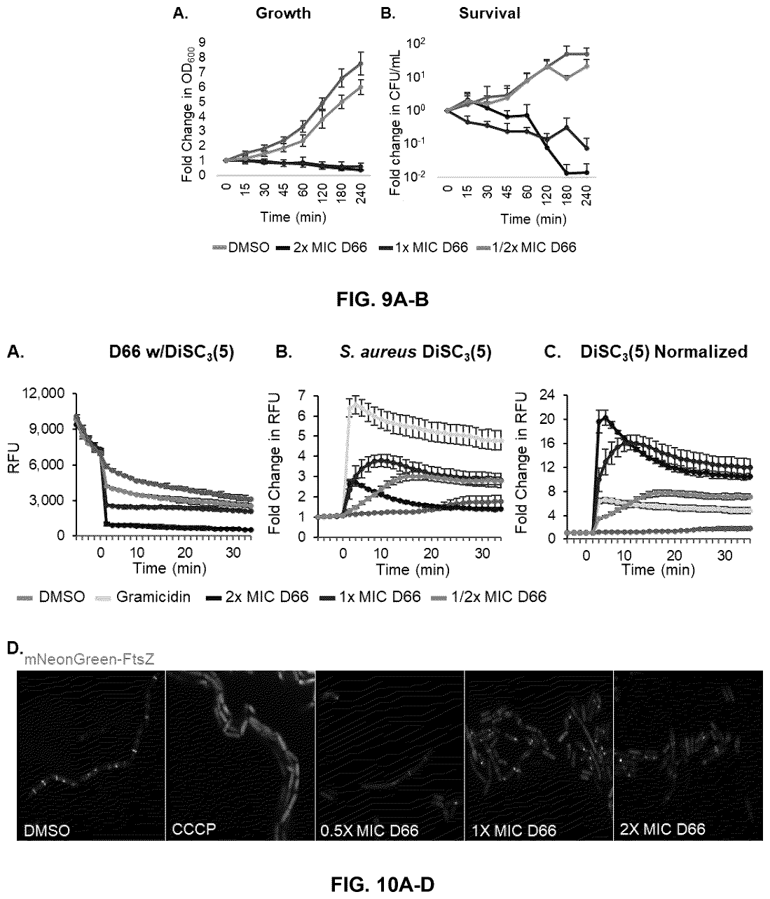

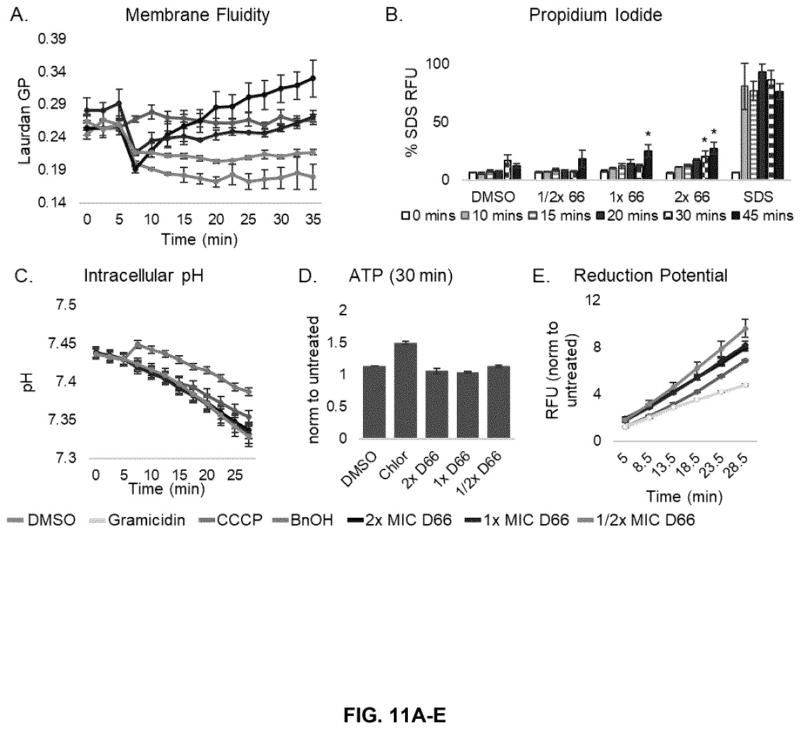

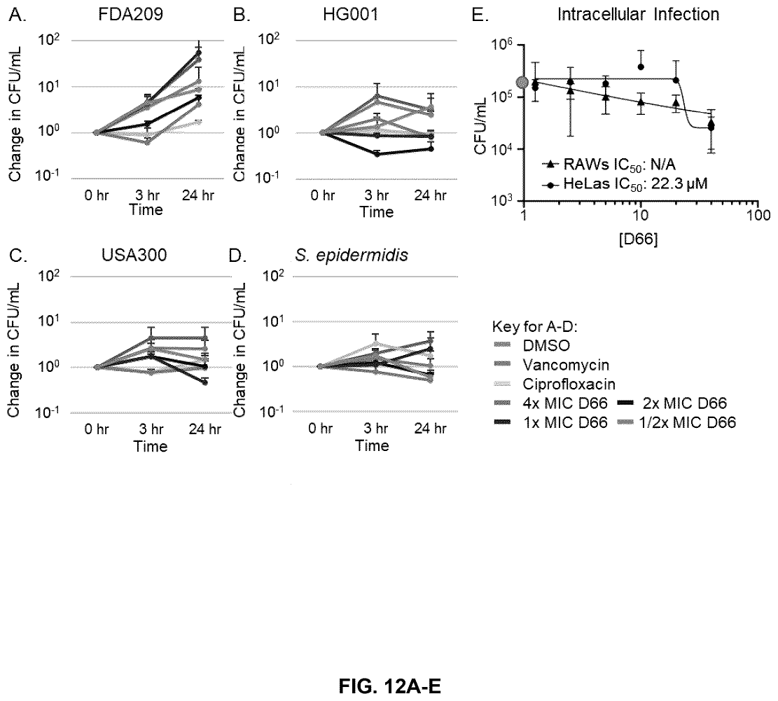

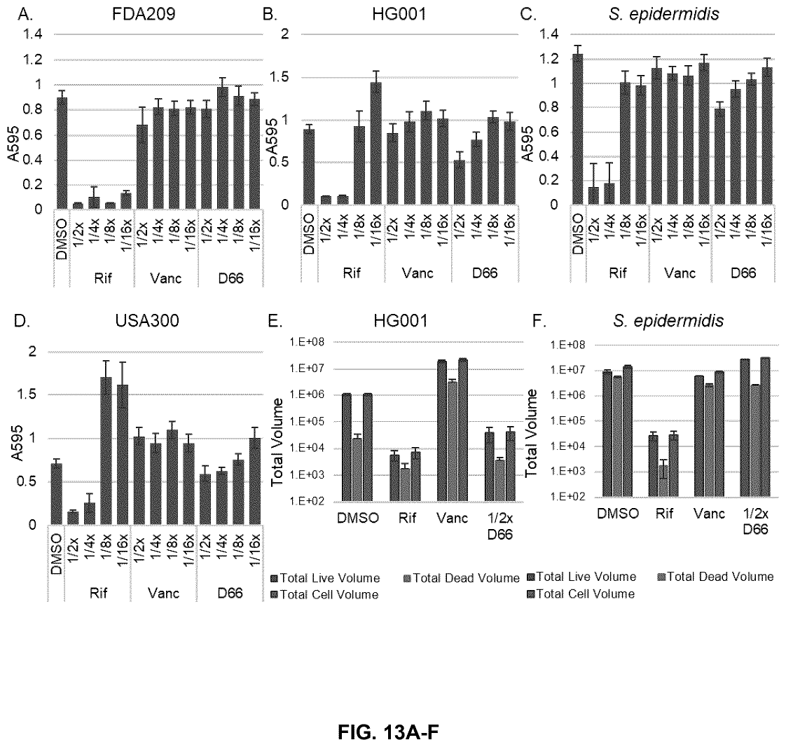

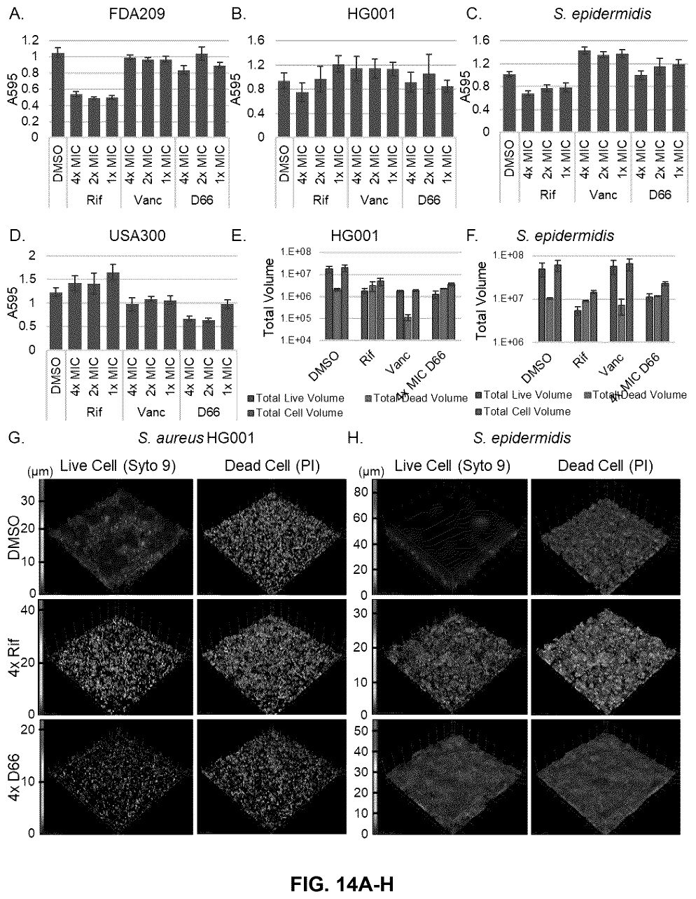

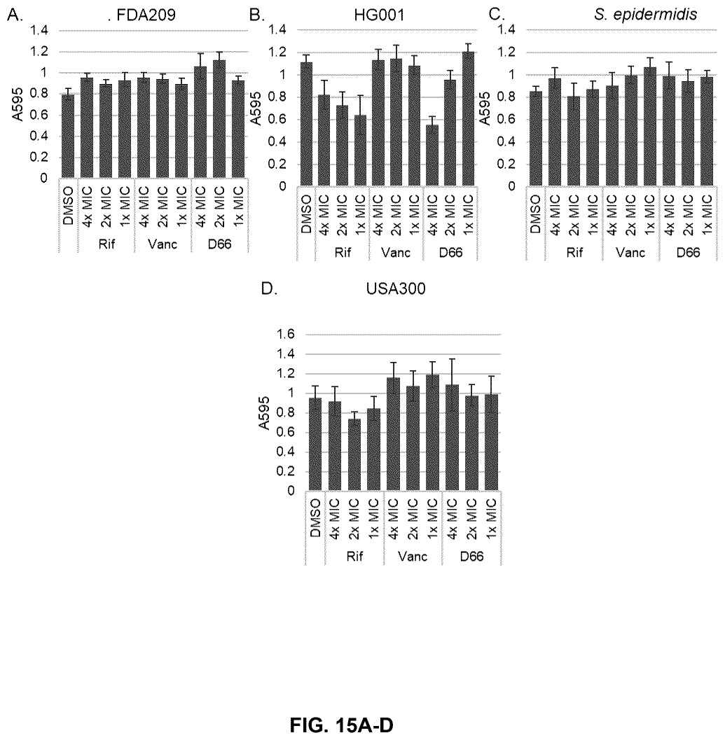

Since D66 is hydrophobic (cLogP=4.73), it could inhibit bacterial growth by affecting the inner membrane. Therefore, Applicants established whether the compound disrupts the proton motive force using the fluorescent probe 3,3′-dipropylthiadicarbocyanine iodide [DiSC3 (5)]. DiSC3 (5) accumulates in membranes that have an electrochemical and proton gradient, where its fluorescence is partially quenched. As a control, Applicants monitored DiSC3 (5) fluorescence in the presence of D66 in cell-free medium and noted that D66 quenches DiSC3 (5) signal in a concentration-dependent manner (FIG. 6A). To enable DiSC3 (5) and D66 to traverse the outer membrane, S. Typhimurium cells were grown in LB with PMB [0.5 μg/mL] and treated with DMSO, JD1, or D66. As expected, JD1 increased DiSC3 (5) fluorescence (FIG. 3A, 6B). Once Applicants had controlled for the quenching effect of D66 on DiSC3 (5) fluorescence, it became apparent that D66 exposure rapidly increased the fluorescence of DiSC3 (5) in a dose-dependent manner (FIG. 3A). These data indicate that the compound disrupts voltage across the bacterial inner membrane.

At least two activities of D66 could rapidly disrupt voltage, physical membrane perturbation and subsequent permeabilization, or depolarization. To determine whether D66 permeabilizes the inner membrane under the same outer membrane-permeabilizing conditions (LB with PMB [0.5 μg/mL]), Applicants used the cell impermeant dye propidium iodide (PI), which enters the cell upon inner membrane damage. After 10 minutes of treatment with 2x MIC95 D66 or the SDS positive control, PI signal increased, indicating it had crossed the inner membrane and bound DNA (FIG. 3B). However, at 1x MIC95, 45 minutes elapsed prior to an increase in PI fluorescence. Thus, D66 disrupts membrane voltage immediately even at 1/2x MIC95, but higher concentrations and longer incubation periods are required to permeabilize the inner membrane.

Another measure of membrane permeabilization is cell lysis. However, in cells grown under outer-membrane perturbing conditions, treatment with D66 at 2x MIC did not reduce the absorbance nor the CFU of bacteria, and instead the cells grew normally in LB (FIG. 3 C) or MHB (FIG. 3 D). These data indicate that significant lysis did not occur in either medium over the course of 18 hours. D66 therefore rapidly disrupts bacterial membrane voltage without permeabilizing or lysing cells, and the bacteria can recover voltage and growth. A primary mechanism of D66 activity therefore appears to be depolarization, and with time and/or higher concentrations, energetic loss and/or compound accumulation permeabilize membranes without lysing cells.

### Example 6: Genetic lesions in the hns gene correlate with resistance to D66 and reduced fitness in macrophages

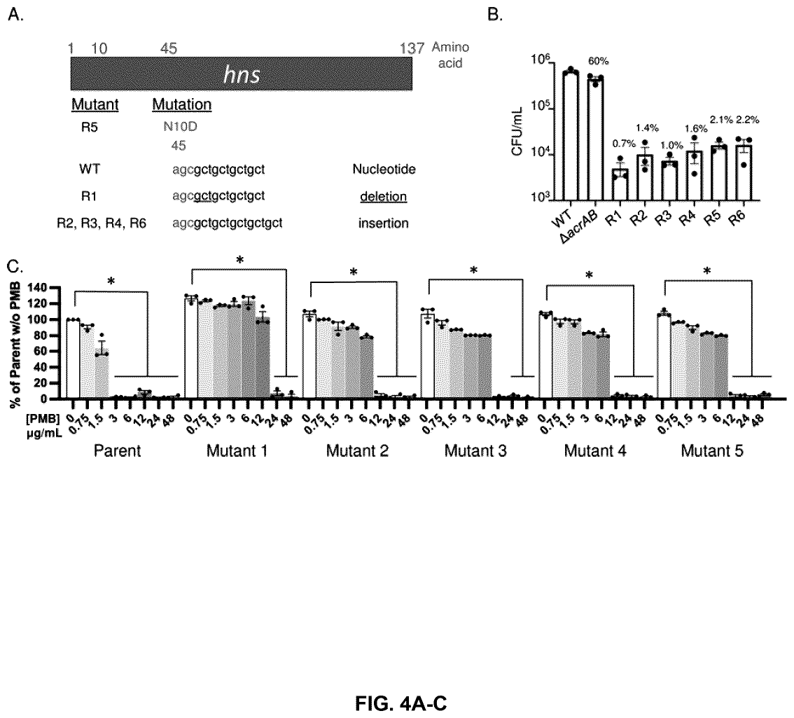

Applicants established whether strains resistant to D66 could be obtained first in a genetic background lacking acrAB, because deletion of this locus sensitized bacteria to the compound (FIG. 2B, C), suggesting that D66 could be an AcrAB-TolC substrate and that selection for mutants in an AacrAB background would increase the probability of obtaining resistant mutants at other loci. Applicants considered selecting for mutants in a AtolC background, but AtolC mutant strains are more severely attenuated than AacrAB strains, and it would be difficult to test recovered mutant strains for resistance to D66 in macrophages. Six independent isolates were evolved in an Aacr AB background in the presence of increasing concentrations of D66, starting at 0.25× MIC and continuing stepwise until growth at 2x MIC was achieved over approximately eight passages. Analysis of whole-genome sequences revealed that all six resistant strains had acquired mutations in the dimerization domain of H-NS that were absent in vehicle-treated control strains (FIG. 4A). The hns mutations are predicted to diminish H-NS function, and loss-of-function mutations in hns enable the expression of efflux pumps, including acrEF, acrD, mdtEF, macAB, and emrKY, which could export D66. H—NS is required for S. Typhimurium virulence in mice, which correlates with replication within macrophages. Consistent with these observations, the D66-resistant mutants survived poorly in macrophages, compared to the parent AacrAB mutant, which, as expected, accumulated to 60% of wild-type levels (FIG. 4B). These results indicate that the resistant strains have decreased fitness during infection, prohibiting the testing of the mutants for D66 resistance in macrophages. Overall, the data show that H-NS contributes to D66 sensitivity, potentially by repressing efflux pumps.

### Example 7: Selection for resistance mutants in sub-inhibitory concentrations of PMB with D66 yielded mutants with increased PMB resistance

Applicants next established whether D66 resistant clones could be obtained in the presence of a subinhibitory concentration of PMB [0.4 μg/mL] and increasing concentrations of D66 up to 3× MIC95. Six independent isolates were obtained. One of the isolates was set aside because resistance was not heritable. Five of the isolates were genetically resistant to the combination of PMB and D66. However, growth assays with the five resistant clones revealed significantly increased resistance to PMB (FIG. 4C). The ease with which resistance to PMB was obtained is consistent with observations that multiple overlapping genetic pathways maintain outer membrane integrity and affect PMB resistance. These data further confirm that a robust outer membrane normally protects bacterial cells from D66.

### Example 8: D66 hyperpolarizes mitochondrial membranes but does not permeabilize host cell membranes

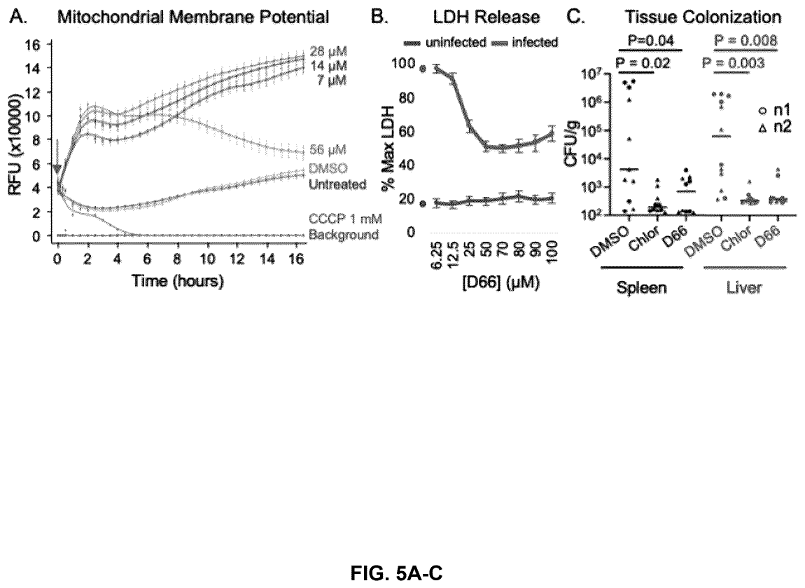

Mitochondrial membranes are similar in lipid composition to bacterial inner membranes in that they contain phosphatidylglycerol and cardiolipin and may therefore be vulnerable to D66. To establish whether D66 alters the voltage of mitochondrial membranes in uninfected macrophages, Applicants used the fluorescent dye tetramethyl rhodamine (TMRM). TMRM accumulates in the mitochondrial inner membrane and increases fluorescence in response to increased membrane potential. RAW 264.7 cells were pre-loaded with TMRM and treated with DMSO, the protonophore carbonyl cyanide m-chlorophenyl hydrazone (CCCP), or D66. As anticipated, CCCP decreased TMRM fluorescence, reflecting membrane depolarization at concentrations effective in SAFIRE (7 μM) (FIG. 5A). Treatment with D66 increased TMRM signal in a dose-dependent manner for the first two hours, suggesting membrane hyperpolarization, an indicator of cell stress. Over time, samples treated with the highest concentration of D66 (56 μM) underwent a steady decline in signal, possibly reflecting compound aggregation and clearance. Mitochondria, therefore, appear to respond modestly to treatment of cells with D66.

To determine whether D66 permeabilizes mammalian cell membranes, Applicants used a standard lactate dehydrogenase release assay (LDH) to monitor membrane leakage. In uninfected RAW 264.7 cells, treatment with D66 had little effect on LDH release (FIG. 5B). Exposure to pathogens radically changes the biology of mammalian cells, so Applicants also measured LDH release in infected RAW 264.7 cells. D66 reduced the percentage of cells that released LDH in a dose-dependent manner (FIG. 5B), consistent with the ability of the compound to reduce bacterial colonization and thereby improve macrophage viability. These data suggest that D66 is minimally toxic to mammalian cell membranes.

### Example 9: D66 reduces bacterial tissue colonization in mice

Since D66 had modest effects on host cells, Applicants evaluated gross toxicity and pharmacokinetics in mice following a single dose. No adverse effects (hunching, tachypnea, or abnormal ambulation) were observed at an intraperitoneal dose of 50 mg/kg after 24 hours. The peak serum concentration observed was 3.5 μM, within range of the 6 μM that is effective in SAFIRE (FIG. 7). The elimination half-life for D66 was estimated to be 3 hours with extensive distribution to tissues based on steady state volume of distribution (Vss) of 43.3 L/kg. Under these conditions, D66 appears to be minimally toxic to mice and is present at levels compatible with testing for potency in vivo.

To establish whether D66 treatment affects S. Typhimurium colonization of tissues in mice, Applicants inoculated C57Bl/6 mice intraperitoneally with 1×104 wild-type bacteria and then treated with 50 mg/kg of D66 intraperitoneally at 10 minutes and 24 hours post-inoculation. All mice that received D66 survived in good condition out to 48 hours, at which time the spleen and liver were harvested. Enumeration of tissue CFU revealed that treatment with D66 reduced S. Typhimurium colonization in both tissues (P<0.05, Mann-Whitney; FIG. 5C). Thus, the compound was tolerated in vivo and had antibacterial potency.

### Example 10: Materials and Methods

Bacterial Strains: S. Typhimurium (SL1344), S. Typhimurium (14028s, ATCC), S. Typhimurium ΔacrAB (ALR1257), E. coli (K-12 derivative BW25113 (wild-type)), E. coli K-12 AtolC (JP313 delta tolC; also called AD3644 and JLD1285).

Media and reagents: Unless otherwise stated, bacteria were grown in LB at 37° C. with aeration. D66 is AW00798 from MolPort. To obtain mid-log phase cells, bacteria were grown overnight in LB, diluted the next morning 1:100 in fresh LB, and then grown to an OD600 of 0.4-0.6). Cation-adjusted MHB was purchased from Sigma-Aldrich (90922).

SAFIRE and CFU assays: SAFIRE assays were performed with RAW 264.7 (TIB-71) macrophages seeded at 5×104 in 100 μL of complete DMEM in 96-well tissue culture plates (Greiner, 655180) and incubated at 37° C. with 5% CO2. S. Typhimurium (SL1344 with sifB: gfp) was grown overnight in LB and diluted to 3×10⁷ CFU/mL in complete DMEM. Twenty-four hours after seeding, 50 μL of bacterial cultures were added to each cell culture well, an approximate multiplicity of infection (MOI) of 30 bacteria to one RAW 264.7 cell. Plates were centrifuged at 500×g for 2 minutes to synchronize the infection. Forty-five minutes after infection, 50 μL of DMEM containing 160 μg/mL gentamicin (Sigma-Aldrich) was added for a final gentamicin concentration of 40 μg/mL. At two hours after infection, cells were treated with vehicle (DMSO) or D66. At 17.5 hours after infection, PBS containing MitoTracker Red CMXRos (Life Technologies) was added to a final concentration of 100 nM. At 18 hours after infection, 16% paraformaldehyde was added to a final concentration of 4% and incubated at room temperature for 15 minutes. Cells were washed, stained with 1 μM DAPI and stored in 90% glycerol in PBS until imaging. After 16 hours of treatment, samples were imaged on a spinning disk confocal microscope, and a MATLAB algorithm calculated bacterial accumulation (GFP fluorescence) within macrophages, as defined by DAPI (DNA) and MitoTracker Red, a vital dye for mitochondrial voltage. GFP+macrophage area is defined as the number of GFP-positive pixels per macrophage divided by the total number of pixels per macrophage, averaged across all macrophages in the field.

CFU assays were performed with RAW 264.7 cells seeded as above or with Hela cells (ATCC CCL-2) seeded at 1x104 cells per 96-well. Cells were infected as described above with either S. Typhimurium SL1344 or 14028, as indicated at an approximate MOI of 30 bacteria per RAW 264.7 cell and 150 bacteria per HeLa cell. Plates were centrifuged and gentamicin treated as above. At 18 hours after infection, wells were washed twice with PBS and lysed with 30 μL 0.1% Triton X-100 in PBS for five minutes. Lysed cells were plated to L-agar and enumerated for CFU.

Minimum inhibitory concentration determination: Overnight LB-grown cultures were diluted in LB or MHB to an optical density (OD₆₀₀) of 0.01 and distributed into polystyrene 96-well flat-bottom plates (Greiner, 655185). D66 was added at concentrations up to 150 μM, near the limit of solubility. The final DMSO concentration was at or below 2%. Where indicated, PMB [0.5 μg/mL] was added prior to D66. Plates were grown at 37° C. with shaking and OD₆₀₀ was monitored (BioTek Synergy H1 or BioTek Eon). MICs were defined as the concentration at which 95% of growth was inhibited (OD₆₀₀).

Novobiocin potentiation assays: Overnight LB-grown cultures were diluted in LB to an optical density at 600 nm (OD₆₀₀) of 0.01 and distributed into polystyrene 96-well flat-bottom plates (Greiner, 655185). D66 was added at concentrations up to 150 μM, near the limit of solubility, and novobiocin was added up to a concentration of 100 μg/mL. The final DMSO concentration was at or below 2%. Plates were grown at 37° C. with shaking for 18 hours and OD₆₀₀ was monitored (BioTek Synergy H1).

Bacterial Membrane potential assays: Membrane potential was measured using the potentiometric fluorescent probe DiSC₃ (5) (Invitrogen). Mid-log phase cells were diluted to an OD₆₀₀ of 0.4. DiSC₃ (5) was added to a final concentration of 2 μM and the culture was incubated at 37° C. in a rotator for 15 minutes. Cells were captured on a 0.45 μm Metricel® membrane filter (Pall), resuspended in fresh LB with 0.5 μg/mL PMB (to enable DiSC₃ (5) and D66 to traverse the outer membrane), and distributed (200 μL) into black polystyrene 96-well plates (Greiner, 655076). Plates were monitored (ex650/em680 nm) on a BioTek Synergy H1 plate reader. After baseline fluorescence was recorded, compound was added to the desired final concentration and measurements were recorded for an additional 30 minutes. This assay was not performed in the BW25113 ΔtolC strain given the difficulty of loading DiSC₃ (5) into the inner membrane of the this strain.

Propidium iodide membrane barrier assays: Compound, DMSO, or SDS was added to mid-log phase cells to the desired concentration, and cultures were sampled at 0, 10, 15, 20, 30 and 45 minutes. Five minutes before harvesting, PI [10 μg/mL] (Life Technologies) was added. Cells were pelleted, washed twice, resuspended in PBS, and monitored (ex535/em617 nm) using a BioTek Synergy H1 plate reader.

Growth curves and kill curves: Mid-log phase cultures were sampled at time 0 and then compound or vehicle control (DMSO) was added. Cultures were incubated at 37° C. with agitation. At the time intervals indicated, aliquots were monitored for OD₆₀₀ and plated for CFU enumeration. Data for OD₆₀₀ and CFU/mL were normalized to time 0.

Evolution of resistant mutants and genetic analysis: To ensure that all isolates started with the same genetic background, a single colony of wild-type S. Typhimurium was resuspended and then distributed into six independent M9 low magnesium broth cultures containing 0.25× MIC of D66. Each day growth was visible, cultures were diluted 1:100 into fresh medium containing an additional 0.25× MIC D66 until growth at 2x MIC was achieved (˜8 passages). Isolates were recovered on LB agar and tested for heritable resistance with 2x MIC D66. Genomic DNA from overnight cultures of resistant mutants and two solvent-treated controls from the same single colony were extracted with the E.Z.N.A® bacterial DNA kit (Omega Bio-tek). Library preparation (Nextera XT) and sequencing (MiSeq V2 2x150 paired end) was performed by the BioFrontiers Sequencing Facility at the University of Colorado Boulder. Data were analyzed for mutations using Snippy (github.com/tseemann/snippy).

Mitochondrial membrane determination with TMRM: Experiments were performed with RAW 264.7 cells between passages one and six. Cells were grown in complete DMEM to a confluency of 70-90%. Cells were scraped, washed, resuspended and diluted in complete DMEM to a final concentration of 5x105 cells/mL. Cells (100 μL) were transferred to a 96-well glass bottom plate (0.17 mm, Brooks Life Sciences) and incubated for 23.5 hours at 37° C. with 5% CO2. The medium was exchanged for 100 μL of complete FluoroBrite DMEM with TMRM

nM] and incubated for 30 minutes. The medium was exchanged for 150 μL of complete FluoroBrite DMEM. Cells were imaged on a Yokogawa CellVoyager™ CV1000 Confocal Scanner System with a 20x/0.75NA objective and an environmentally controlled multi-well chamber over 30 minutes with images acquired every 10 minutes. Compounds were added (50 μL) with a multichannel pipet to obtain the desired concentration and a final volume of 200 μL with 0.5% DMSO. Cells were imaged over 16 hours with acquisition every 30 minutes of two fields of view per well. Five images over a z-dimension of 15 μM were sampled per field. The resulting volumes were converted into maximum intensity projections and TMRM foreground signal was extracted via a MATLAB R2018a (MathWorks) script and normalized to time zero for each field.

LDH assays: An LDH-cytotoxicity assay kit (Abcam ab65393) was used according to the manufacturer's instructions. RAW 264.7 cells were seeded and remained uninfected or with infected as described for the CFU assay with SL1344.

Murine pharmacokinetic analyses and infections: Three female C57BL/6 mice were injected intraperitoneally (IP) with 50 mg/kg of D66 formulated in DMSO (50 μL). This dose was selected based on D66 solubility, which suggested the compound distributes evenly within the mouse such that a dose of 54 μg per 20 g mouse is needed to achieve a concentration of approximately 7.8 UM, the IC50 of D66 against S. Typhimurium in macrophages. Three mice were initially dosed and observed for 24 hours to determine tolerability. Following the lack of observable toxicity and gross pathological lesions in liver, kidney, and gastrointestinal tissues, another 12 mice were treated with the same dose of D66 and plasma samples collected at 0.5,1, 4, and 8 hours by cardiac exsanguination under isoflurane anesthesia. Plasma D66 levels were measured using a liquid chromatography coupled to tandem mass spectrometry (LC/MS/MS) assay by the University of Colorado Cancer Center Drug Discovery and Development Shared Resource. The assay used monitored the transition of D66 (376 m/z->176 m/z) and was linear from 1-1000 ng/ml with an accuracy and precision of 90.3%+10.4% (% CV) based on quality control (QC) samples included with analyzed unknown samples. The peak serum concentration observed was 3.5 μM, and the terminal half-life was 3.0 hours.

Female C57Bl/6 7-8-week-old mice were IP inoculated with S. Typhimurium strain SL1344. Six mice per cohort were IP-treated with 100 μL of vehicle (50% DMSO), 50 mg/kg of chloramphenicol, or 50 mg/kg of D66 at 10 minutes and 24 hours post-infection. Mice were euthanized at 48 hours by CO2 asphyxiation, followed by cervical dislocation [7]. Spleen and liver were collected, homogenized in 1 mL PBS and serially diluted for plating to enumerate CFU. The experiment was performed twice independently with 7×10³ CFU, 3×104 CFU respectively, as determined by plating. A ROUT test for outliers and a Mann-Whitney test for significance were performed in GraphPad Prism.

### REFERENCES

1. Miller SI, Salama NR. The gram-negative bacterial periplasm: Size matters. PLOS Biol [Internet]. 2018 Jan. 17 [cited 2020 Jan. 17]; 16 (1): e2004935.
2. Silhavy TJ, Kahne D, Walker S. The bacterial cell envelope. Cold Spring Harb Perspect Biol. 2010 May;2 (5): a000414.
3. Simpson BW, Trent MS. Pushing the envelope: LPS modifications and their consequences. Nat Rev Microbiol. 2019 July; 17 (7): 403-16.
4. Matamouros S, Miller SI. S. Typhimurium strategies to resist killing by cationic antimicrobial peptides. Biochim Biophys Acta. 2015 November;1848 (11 Pt B): 3021-5.
5. Demchick P, Koch AL. The permeability of the wall fabric of Escherichia coli and Bacillus subtilis. J Bacteriol. 1996 February; 178 (3): 768-73.
6. Egan AJF, Errington J, Vollmer W. Regulation of peptidoglycan synthesis and remodelling. Nat Rev Microbiol. 2020 August; 18 (8): 446-60.
7. Zgurskaya HI, Rybenkov VV. Permeability barriers of Gram-negative pathogens. Ann N Y Acad Sci. 2020 January; 1459 (1): 5-18.
8. Klenotic PA, Moseng MA, Morgan CE, Yu EW. Structural and Functional Diversity of Resistance-Nodulation-Cell Division Transporters. Chem Rev. 2020 Nov. 19;
9. Heesterbeek DAC, Muts RM, van Hensbergen VP, de Saint Aulaire P, Wennekes T, Bardoel

BW, et al. Outer membrane permeabilization by the membrane attack complex sensitizes Gram-negative bacteria to antimicrobial proteins in serum and phagocytes. PLOS Pathog. 2021 Jan. 22;17 (1): e1009227.
10. Lim Y, Shiver AL, Khariton M, Lane KM, Ng KM, Bray SR, et al. Mechanically resolved imaging of bacteria using expansion microscopy. PLOS Biol. 2019 October; 17 (10): e3000268.
11. Kutsch M, Sistemich L, Lesser CF, Goldberg M B, Herrmann C, Coers J. Direct binding of polymeric GBP1 to LPS disrupts bacterial cell envelope functions. EMBO J. 2020 Jul. 1;39 (13): e104926.
12. Kutsch M, Coers J. Human Guanylate Binding Proteins: nanomachines orchestrating host defense. FEBS J. 2020 Dec. 12;
13. Heesterbeek D a. C, Martin N I, Velthuizen A, Duijst M, Ruyken M, Wubbolts R, et al. Complement-dependent outer membrane perturbation sensitizes Gram-negative bacteria to Gram-positive specific antibiotics. Sci Rep. 2019 Feb. 28;9 (1): 3074.
14. Guina T, Yi E C, Wang H, Hackett M, Miller SI. A PhoP-regulated outer membrane protease of Salmonella enterica serovar typhimurium promotes resistance to alpha-helical antimicrobial peptides. J Bacteriol. 2000 July; 182 (14): 4077-86.
15. Kawai Y, Mickiewicz K, Errington J. Lysozyme Counteracts B-Lactam Antibiotics by Promoting the Emergence of L-Form Bacteria. Cell. 2018 Feb. 22;172 (5): 1038-1049.e10.
16. Kumaraswamy M, Lin L, Olson J, Sun C F, Nonejuie P, Corriden R, et al. Standard susceptibility testing overlooks potent azithromycin activity and cationic peptide synergy against MDR Stenotrophomonas maltophilia. J Antimicrob Chemother. 2016 May;71 (5): 1264-9.
17. Lin L, Nonejuie P, Munguia J, Hollands A, Olson J, Dam Q, et al. Azithromycin Synergizes with Cationic Antimicrobial Peptides to Exert Bactericidal and Therapeutic Activity Against Highly Multidrug-Resistant Gram-Negative Bacterial Pathogens. EBioMedicine [Internet]. 2015 July [cited 2016 Jun. 14];2 (7): 690-8.
18. Nagy T A, Crooks A L, Quintana JLJ, Detweiler C S. Clofazimine Reduces the Survival of Salmonella enterica in Macrophages and Mice. ACS Infect Dis [Internet]. 2020 May 8 [cited 2020 Jun. 1];6 (5): 1238-49.
19. Dombach J L, Quintana JLJ, Nagy T A, Wan C, Crooks A L, Yu H, et al. A small molecule that mitigates bacterial infection disrupts Gram-negative cell membranes and is inhibited by cholesterol and neutral lipids. PLOS Pathog. 2020 Dec. 8;16 (12): e1009119.
20. Reens A L, Crooks A L, Su C C, Nagy T A, Reens D L, Podoll JD, et al. A cell-based infection assay identifies efflux pump modulators that reduce bacterial intracellular load. PLOS Pathog. 2018 Jun. 7;14 (6): e1007115.
21. Detweiler C S. Infection-based chemical screens uncover host-pathogen interactions. Curr Opin Microbiol. 2020 Feb. 6; 54:43-50.
22. Mueller J H, Hinton J. A Protein-Free Medium for Primary Isolation of the Gonococcus and Meningococcus. Proc Soc Exp Biol Med [Internet]. 1941 Oct. 1 [cited 2019 Jul. 26];48 (1): 330-3.
23. Bertani G. Lysogeny at Mid-Twentieth Century: P1, P2, and Other Experimental Systems. J Bacteriol [Internet]. 2004 Feb. 1 [cited 2019 Jul. 27]; 186 (3): 595-600.
24. Bertani G. Studies on lysogenesis. I. The mode of phage liberation by lysogenic Escherichia coli. J Bacteriol. 1951 September;62 (3): 293-300.
25. Nagy T A, Quintana JLJ, Reens A L, Crooks A L, Detweiler C S. Autophagy Induction by a Small Molecule Inhibits Salmonella Survival in Macrophages and Mice. Antimicrob Agents Chemother [Internet]. 2019 Dec. 1 [cited 2020 Apr. 9];63 (12): 1-16.
26. Ofek I, Cohen S, Rahmani R, Kabha K, Tamarkin D, Herzig Y, et al. Antibacterial synergism of polymyxin B nonapeptide and hydrophobic antibiotics in experimental gram-negative infections in mice. Antimicrob Agents Chemother. 1994 February;38 (2): 374-7.
27. Vaara M, Vaara T. Sensitization of Gram-negative bacteria to antibiotics and complement by a nontoxic oligopeptide. Nature. 1983 Jun. 9;303 (5917): 526-8.
28. Katz M, Tsubery H, Kolusheva S, Shames A, Fridkin M, Jelinek R. Lipid binding and membrane penetration of polymyxin B derivatives studied in a biomimetic vesicle system. Biochem J. 2003 Oct. 15;375 (Pt 2): 405-13.
29. Sahalan A Z, Dixon R A. Role of the cell envelope in the antibacterial activities of polymyxin B and polymyxin B nonapeptide against Escherichia coli. Int J Antimicrob Agents. 2008 March;31 (3): 224-7.
30. Dombach J L, Quintana JLJ, Detweiler C S. Staphylococcal Bacterial Persister Cells, Biofilms, and Intracellular Infection Are Disrupted by JD1, a Membrane-Damaging Small Molecule. mBio. 2021 Oct. 12;e0180121.
31. the Winkel J D, Gray D A, Seistrup K H, Hamoen L W, Strahl H. Analysis of Antimicrobial-Triggered Membrane Depolarization Using Voltage Sensitive Dyes. Front Cell Dev Biol [Internet]. 2016 Apr. 13 [cited 2019 Jul. 21];4.
32. Nishino K, Latifi T, Groisman E A. Virulence and drug resistance roles of multidrug efflux systems of Salmonella enterica serovar Typhimurium. Mol Microbiol [Internet]. 2006 Jan. 1 [cited 2016 Aug. 15];59 (1): 126-41.
33. Williams R M, Rimsky S, Buc H. Probing the structure, function, and interactions of the Escherichia coli H-NS and StpA proteins by using dominant negative derivatives. J Bacteriol. 1996 August; 178 (15): 4335-43.
34. Ueguchi C, Seto C, Suzuki T, Mizuno T. Clarification of the dimerization domain and its functional significance for the Escherichia coli nucleoid protein H-NS. J Mol Biol. 1997 Nov. 28;274 (2): 145-51.
35. Grainger D C. Structure and function of bacterial H-NS protein. Biochem Soc Trans. 2016 Dec. 15;44 (6): 1561-9.
36. Cho H J, Misra R. Mutational Activation of Antibiotic Resistant Mechanisms in the Absence of Major Drug Efflux Systems of Escherichia coli. J Bacteriol. 2021 May 10;
37. Hirata T, Saito A, Nishino K, Tamura N, Yamaguchi A. Effects of efflux transporter genes on susceptibility of Escherichia coli to tigecycline (GAR-936). Antimicrob Agents Chemother. 2004 June;48 (6): 2179-84.
38. Harrison J A, Pickard D, Higgins C F, Khan A, Chatfield S N, Ali T, et al. Role of hns in the virulence phenotype of pathogenic salmonellae. Mol Microbiol. 1994 July;13 (1): 133-40.
39. Buchmeier N A, Heffron F. Intracellular survival of wild-type Salmonella typhimurium and macrophage-sensitive mutants in diverse populations of macrophages. Infect Immun. 1989 January;57 (1): 1-7.
40. Tamayo R, Ryan S S, McCoy A J, Gunn J S. Identification and genetic characterization of PmrA-regulated genes and genes involved in polymyxin B resistance in Salmonella enterica serovar typhimurium. Infect Immun. 2002 December;70 (12): 6770-8.
41. Tran A X, Lester M E, Stead C M, Raetz C R H, Maskell D J, McGrath SC, et al. Resistance to the antimicrobial peptide polymyxin requires myristoylation of Escherichia coli and Salmonella typhimurium lipid A. J Biol Chem. 2005 Aug. 5;280 (31): 28186-94.
42. Guo L, Lim K B, Gunn J S, Bainbridge B, Darveau R P, Hackett M, et al. Regulation of lipid A modifications by Salmonella typhimurium virulence genes phoP-phoQ. Science [Internet]. 1997 Apr. 11 [cited 2009 Mar. 2];276 (5310): 250-3.
43. Detweiler C S, Monack D M, Brodsky I E, Mathew H, Falkow S. virk, somA and rcsC are important for systemic Salmonella enterica serovar Typhimurium infection and cationic peptide resistance. Mol Microbiol. 2003 April;48 (2): 385-400.
44. Epand R M, Epand R F. Domains in bacterial membranes and the action of antimicrobial agents. Mol Biosyst. 2009 June;5 (6): 580-7.
45. Leonard A P, Cameron R B, Speiser J L, Wolf B J, Peterson Y K, Schnellmann RG, et al. Quantitative analysis of mitochondrial morphology and membrane potential in living cells using high-content imaging, machine learning, and morphological binning. Biochim Biophys Acta. 2015 February;1853 (2): 348-60.
46. Sieprath T, Corne T D J, Nooteboom M, Grootaert C, Rajkovic A, Buysschaert B, et al. Sustained accumulation of prelamin A and depletion of lamin A/C both cause oxidative stress and mitochondrial dysfunction but induce different cell fates. Nucl Austin Tex. 2015;6 (3): 236-46.
47. Giovannini C, Matarrese P, Scazzocchio B, Sanchez M, Masella R, Malorni W. Mitochondria hyperpolarization is an early event in oxidized low-density lipoprotein-induced apoptosis in Caco-2 intestinal cells. FEBS Lett. 2002 Jul. 17;523 (1-3): 200-6.
48. Mick E, Titov D V, Skinner O S, Sharma R, Jourdain A A, Mootha V K. Distinct mitochondrial defects trigger the integrated stress response depending on the metabolic state of the cell. eLife. 2020 May 28;9: e49178.
49. Fotakis G, Timbrell J A. In vitro cytotoxicity assays: comparison of LDH, neutral red, MTT and protein assay in hepatoma cell lines following exposure to cadmium chloride. Toxicol Lett. 2006 Jan. 5;160 (2): 171-7.
50. Korzeniewski C, Callewaert D M. An enzyme-release assay for natural cytotoxicity. J Immunol Methods. 1983 Nov. 25;64 (3): 313-20.
51. Srikumar S, Kröger C, Hébrard M, Colgan A, Owen S V, Sivasankaran SK, et al. RNA-seq Brings New Insights to the Intra-Macrophage Transcriptome of Salmonella Typhimurium. PLOS Pathog. 2015;11 (11): e1005262.
52. Padilla E, Llobet E, Doménech-Sánchez A, Martínez-Martínez L, Bengoechea J A, Albertí S. Klebsiella pneumoniae AcrAB efflux pump contributes to antimicrobial resistance and virulence. Antimicrob Agents Chemother. 2010 January;54 (1): 177-83.
53. Kus J V, Gebremedhin A, Dang V, Tran S L, Serbanescu A, Barnett Foster D. Bile salts induce resistance to polymyxin in enterohemorrhagic Escherichia coli 0157: H7. J Bacteriol. 2011 September; 193 (17): 4509-15.
54. Gunn J S, Lim K B, Krueger J, Kim K, Guo L, Hackett M, et al. PmrA-PmrB-regulated genes necessary for 4-aminoarabinose lipid A modification and polymyxin resistance. Mol Microbiol [Internet]. 1998 Mar. 1 [cited 2016 May 27];27 (6): 1171-82.
55. Liu J, Huang Z, Ruan B, Wang H, Chen M, Rehman S, et al. Quantitative proteomic analysis reveals the mechanisms of polymyxin B toxicity to Escherichia coli. Chemosphere. 2020 November;259:127449.
56. To T L, Cuadros A M, Shah H, Hung W H W, Li Y, Kim SH, et al. A Compendium of Genetic Modifiers of Mitochondrial Dysfunction Reveals Intra-organelle Buffering. Cell. 2019 Nov. 14;179 (5): 1222-1238.e17.
57. Brand M D, Nicholls D G. Assessing mitochondrial dysfunction in cells. Biochem J. 2011 Apr. 15;435 (2): 297-312.
58. Reens A L, Crooks A L, Su C C, Nagy T A, Reens D L, Podoll JD, et al. A cell-based infection assay identifies efflux pump modulators that reduce bacterial intracellular load. Miller SI, editor. PLOS Pathog. 2018 June;14 (6): e1007115.
59. Ellis M J, Tsai C N, Johnson J W, French S, Elhenawy W, Porwollik S, et al. A macrophage-based screen identifies antibacterial compounds selective for intracellular Salmonella Typhimurium. Nat Commun. 2019 14;10 (1): 197.
60. Lieberman L A, Higgins D E. A small-molecule screen identifies the antipsychotic drug pimozide as an inhibitor of Listeria monocytogenes infection. Antimicrob Agents Chemother. 2009 February;53 (2): 756-64.
61. Stanley S A, Barczak A K, Silvis M R, Luo S S, Sogi K, Vokes M, et al. Identification of host-targeted small molecules that restrict intracellular Mycobacterium tuberculosis growth. PLOS Pathog. 2014 February; 10 (2): e1003946.
62. Hoiseth S K, Stocker B a. D. Aromatic-dependent Salmonella typhimurium are non-virulent and effective as live vaccines. Nature [Internet]. 1981 May 21 [cited 2017 Jul. 13];291 (5812): 238-9.
63. Grenier F, Matteau D, Baby V, Rodrigue S. Complete Genome Sequence of Escherichia coli BW25113. Genome Announc. 2014 Oct. 16;2 (5): e01038-14.
64. Montaño ET, Nideffer J F, Sugie J, Enustun E, Shapiro A B, Tsunemoto H, et al. Bacterial Cytological Profiling Identifies Rhodanine-Containing PAINS Analogs as Specific Inhibitors of Escherichia coli Thymidylate Kinase In Vivo. J Bacteriol. 2021 Sep. 8;203 (19): e0010521.
65. Rollenhagen C, Sörensen M, Rizos K, Hurvitz R, Bumann D. Antigen selection based on expression levels during infection facilitates vaccine development for an intracellular pathogen. Proc Natl Acad Sci USA. 2004 Jun. 8;101 (23): 8739-44.

TABLE 1
Concentrations of D66 that inhibit S. Typhimurium and E. coli growth

| | IC₅₀ ¹ in | MIC₅₀² | | cMIC₉₅³ | MIC₅₀ | cMIC₉₅³ |
|---|---|---|---|---|---|---|
| Species (strain) | SAFIRE | in LB | | in LB | in MHB | in MHB |
| Condition/mutation | μM | μM | μg/mL | μM | μM | μM |
| S. Typhimurium (SL1344) | 6.0 ± 0.5⁴ | >150 | >57 | >150 | >150 | >150 |
| w/PMB (0.5 ug/mL) | NA | 45 ± 4 | 17 | 54 | 40 ± 1 | 46 |
| ΔacrAB | NA | 128 ± 2 | 48 | 142 | 81 ± 6 | 116 |
| E. coli (K-12) | NA | >150 | >57 | >150 | >150 | >150 |
| ΔtolC | NA | 47 ± 1 | 18 | 52 | 36 ± 1 | 43 |

¹ IC₅₀: The concentration of D66 that prevents half of the accumulation of GFP signal in macrophages
²MIC₅₀: The concentration of D66 that prevents half of the growth of the corresponding bacterial strain in broth,
³cMIC₉₅: The calculated concentration of D66 that prevents 95% of growth of the corresponding bacterial strain in broth, derived from the non-linear regression calculated from the MIC curves. These values were used for experiments, as indicated in figure legends.
⁴FIG. 1B, with the SL1344 sifB::gfp strain

1-13. (canceled)

14. A method for disrupting the inner membrane structure of Gram-negative bacterium, comprising contacting the bacterium with a pharmaceutical composition containing a therapeutically effective amount of a compound having a structure according to Formula (I): 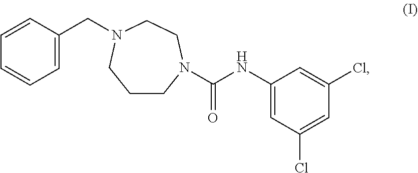 [SMILES: O=C(Nc1cc(Cl)cc(Cl)c1)N1CCCN(Cc2ccccc2)CC1]
and at least one pharmaceutically acceptable carrier.

15. A method for disrupting the voltage across a bacterial cytoplasmic membrane of Gram-positive bacterium, comprising contacting the bacterium with a compound according to Formula (I):

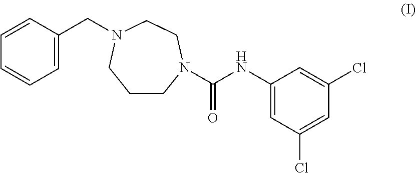 [SMILES: O=C(Nc1cc(Cl)cc(Cl)c1)N1CCCN(Cc2ccccc2)CC1]

16. A method for disrupting the voltage across a bacterial cytoplasmic membrane of Gram-positive bacterium, comprising contacting the bacterium with a pharmaceutical composition containing a therapeutically effective amount of a compound according to Formula (I): 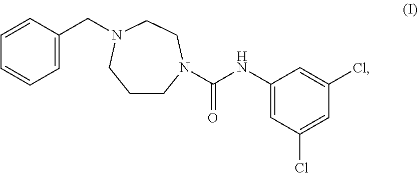 [SMILES: O=C(Nc1cc(Cl)cc(Cl)c1)N1CCCN(Cc2ccccc2)CC1]
and
and at least one pharmaceutically acceptable carrier.

17-20. (canceled)
